(19) United States
(12) Patent Application Publication
(10) Pub. No.: US 2007/0197517 A1
(43) Pub. Date: Aug. 23, 2007

(54) Combination therapy for hyperproliferative disease

(75) Inventors: Jitesh P. Jani, East Lyme, CT (US); Jean S. Beebe, Salem, CT (US); Tracey L. Schaeffer, Franklin, CT (US); Diane I. Healey, Madison, CT (US); Karen J. Ferrante, East Greenwich, RI (US); James J. O'Leary, Mystic, CT (US)

Correspondence Address: PFIZER INC, 10555 SCIENCE CENTER DRIVE, SAN DIEGO, CA, 92121, US

(73) Assignee: Pfizer Inc.

(21) Appl. No.: 10/643,546

(22) Filed: Aug. 19, 2003

Related U.S. Application Data
(60) Provisional application No. 60/404,461, filed on Aug. 19, 2002.

(51) Int. Cl. A61K 31/41; A61K 31/35; A61K 31/53; C07D 239/42

(52) U.S. Cl. 514/229.5; 514/242; 514/256; 514/359; 514/453

(57) ABSTRACT

This invention relates a method of treating hyperproliferative diseases. More particularly, the present invention relates to a method of treating hyperproliferative diseases, such as cancer, comprising the step of administering to a mammal in need of such treatment, either simultaneously or sequentially, (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of an isothiazole derivative. The combinations of the present invention may optionally include an anti-hypertensive agent. This invention also relates to pharmaceutical compositions useful in the treatment of hyperproliferative diseases in mammals, containing such combinations. The present invention also relates to kits having a first compartment with a compound of formula 1 and a second compartment containing a taxane derivative, a platinum coordination complex, a nucleoside analog, an anthracycline, a topoisomerase inhibitor, or an aromatase inhibitor and a third compartment containing an anti-hypertensive agent.

### BACKGROUND OF THE INVENTION

This invention relates a method of treating hyperproliferative diseases. More particularly, the present invention relates to a method of treating hyperproliferative diseases, such as cancer, comprising the step of administering to a mammal in need of such treatment, either simultaneously or sequentially, (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) an isothiazole derivative. The methods of the present invention may optionally include an anti-hypertensive agent. This invention also relates to pharmaceutical compositions useful in the treatment of hyperproliferative diseases in mammals, containing a taxane derivative, a platinum coordination complex, a nucleoside analog, an anthracycline, a topoisomerase inhibitor, or an aromatase inhibitor, in combination with an isothiazole derivative.

Cancer continues to be one of the leading causes of death in the United States and other developed countries. A large number of drugs are currently being tested in clinical trials for the treatment of a wide variety of cancers. One of the preferred approaches to the treatment of cancer has been combination therapy. One of the advantages of combination therapy has been the ability to attack the cancer using agents that have different mechanisms of action. This has been found in some cases to lead to an enhanced efficacy in trials as indicated by improved disease free survival and overall survival from the use of combination protocols.

One of the newer treatments that is been developed is that of target therapy to treat cancer. It is known that a cell may become cancerous by virtue of the transformation of a portion of its DNA into an oncogene (i.e. a gene that upon activation leads to the formation of malignant tumor cells). Many oncogenes encode proteins which are aberrant tyrosine kinases capable of causing cell transformation. Alternatively, the overexpression of a normal proto-oncogenic tyrosine kinase may also result in proliferative disorders, sometimes resulting in a malignant phenotype. It has been shown that certain tyrosine kinases may be mutated or overexpressed in many human cancers such as brain, lung, squamous cell, bladder, gastric, breast, head and neck, oesophageal, gynecological and thyroid cancers. Furthermore, the overexpression of a ligand for a tyrosine kinase receptor may result in an increase in the activation state of the receptor, resulting in proliferation of the tumor cells or endothelial cells. Thus, it is believed that inhibitors of receptor tyrosine kinases, such as the compounds of the present invention, are useful as selective inhibitors of the growth of mammalian cancer cells.

It is known that polypeptide growth factors, such as vascular endothelial growth factor (VEGF) having a high affinity to the human kinase insert-domain-containing receptor (KDR) or the murine fetal liver kinase 1 (FLK-1) receptor, have been associated with the proliferation of endothelial cells and more particularly vasculogenesis and angiogenesis. See PCT international application publication number WO 95/21613 (published Aug. 17, 1995). Agents, such as the compounds of the present invention, that are capable of binding to or modulating the KDR/FLK-1 receptor may be used to treat disorders related to vasculogenesis or angiogenesis such as diabetes, diabetic retinopathy, hemangioma, glioma, melanoma, Kaposi's sarcoma and ovarian, breast, lung, pancreatic, prostate, colon and epidermoid cancer.

### SUMMARY OF THE INVENTION

The present invention relates to a combination of anti-hyperproliferative agents and a method of treating hyperproliferative diseases, such as cancer, comprising the step of administering to a mammal in need of such treatment, either simultaneously or sequentially, (i) a therapeutically effective amount of an isothiazole derivative, and (ii) a therapeutically effective amount of a taxane derivative, a platinum coordination complex, a nucleoside analog, an anthracycline, a topoisomerase inhibitor, or an aromatase inhibitor.

The present invention relates to a method of treating a hyperproliferative disorder in a mammal which comprises administering to said mammal in need of such treatment, either simultaneously or sequentially, (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of a compound of the formula 1

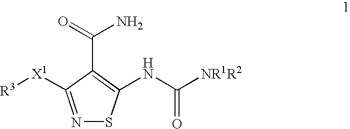

or a pharmaceutically acceptable salt, prodrug or solvate thereof, wherein:

wherein X¹ is O or S;

R¹ is H, C₁-C₁₀ alkyl, C₂-C₁₀ alkenyl, C₂-C₁₀ alkynyl, —C(O)(C₁-C₁₀ alkyl), —(CH₂)ₜ(C₆-C₁₀ aryl), —(CH₂)ₜ(4-10 membered heterocyclic), —C(O)(CH₂)ₜ(C₆-C₁₀ aryl), or —C(O)(CH₂)ₜ(5-10 membered heterocyclic), wherein t is an integer from 0 to 5; said alkyl group optionally includes 1 or 2 hetero moieties selected from O, S and —N(R⁶)— with the proviso that two O atoms, two S atoms, or an O and S atom are not attached directly to each other; said aryl and heterocyclic R¹ groups are optionally fused to a C₆-C₁₀ aryl group, a C₅-C₈ saturated cyclic group, or a 5-10 membered heterocyclic group; 1 or 2 carbon atoms in the foregoing heterocyclic moieties are optionally substituted by an oxo (═O) moiety; the —(CH₂)ₜ— moieties of the foregoing R¹ groups optionally include a carbon-carbon double or triple bond where t is an integer from 2 to 5; and the foregoing R¹ groups, except H, are optionally substituted by 1 to 3 R⁴ groups;

R² is selected from the list of substituents provided in the definition of R¹, —SO₂(CH₂)ₜ(C₆-C₁₀ aryl), —SO₂(CH₂)ₜ(5-10 membered heterocyclic), and —OR⁵, t is an integer ranging from 0 to 5, the —(CH₂)ₜ— moieties of the foregoing R² groups optionally include a carbon-carbon double or triple bond where t is an integer from 2 to 5, and the foregoing R² groups are optionally substituted by 1 to 3 R⁴ groups;

or R¹ and R² may be taken together with the nitrogen to which each is attached to form a 4-10 membered saturated monocyclic or polycyclic ring or a 5-10 membered heteroaryl ring, wherein said saturated and heteroaryl rings optionally include 1 or 2 heteroatoms selected from O, S and —N(R⁶)— in addition to the nitrogen to which R¹ and R² are attached, said —N(R⁶)— is optionally ═N— or —N═ where R¹ and R² are taken together as said heteroaryl group, said saturated ring optionally may be partially unsaturated by including 1 or 2 carbon-carbon double bonds, and said saturated and heteroaryl rings, including the R⁶ group of said —N(R⁶)—, are optionally substituted by 1 to 3 R⁴ groups;

R³ is H, C₁-C₁₀ alkyl, C₂-C₁₀ alkenyl, C₂-C₁₀ alkynyl, —(CH₂)ₜ(C₆-C₁₀ aryl), or —(CH₂)ₜ(5-10 membered heterocyclic), wherein t is an integer from 0 to 5; said alkyl group optionally includes 1 or 2 hetero moieties selected from O, S and —N(R⁶)— with the proviso that two O atoms, two S atoms, or an O and S atom are not attached directly to each other; said aryl and heterocyclic R³ groups are optionally fused to a C₆-C₁₀ aryl group, a C₅-C₈ saturated cyclic group, or a 5-10 membered heterocyclic group; 1 or 2 carbon atoms in the foregoing heterocyclic moieties are optionally substituted by an oxo (═O) moiety; the —(CH₂)ₜ— moieties of the foregoing R³ groups optionally include a carbon-carbon double or triple bond where t is an integer from 2 to 5, and the foregoing R³ groups are optionally substituted by 1 to 5 R⁴ groups;

each R⁴ is independently selected from C₁-C₁₀ alkyl, C₂-C₁₀ alkenyl, C₂-C₁₀ alkynyl, halo, cyano, nitro, trifluoromethyl, trifluoromethoxy, azido, —OR⁵, —C(O)R⁵, —C(O)OR⁵, —NR⁶C(O)OR⁵, —OC(O)R⁵, —NR⁶SO₂R⁵, —SO₂NR⁵R⁶, —NR⁶C(O)R⁵, —C(O)NR⁵R⁶, —NR⁵R⁶, —S(O)ⱼR⁷ wherein j is an integer ranging from 0 to 2, —SO₃H, —NR⁵(CR⁶R⁷)ₜOR⁶, —(CH₂)ₜ(C₆-C₁₀ aryl), —SO₂(CH₂)ₜ(C₆-C₁₀ aryl), —S(CH₂)ₜ(C₆-C₁₀ aryl), —O(CH₂)ₜ(C₆-C₁₀ aryl), —(CH₂)ₜ(5-10 membered heterocyclic), and —(CR⁶R⁷)ₘOR⁶, wherein m is an integer from 1 to 5 and t is an integer from 0 to 5; said alkyl group optionally contains 1 or 2 hetero moieties selected from O, S and —N(R⁶)— with the proviso that two O atoms, two S atoms, or an O and S atom are not attached directly to each other; said aryl and heterocyclic R⁴ groups are optionally fused to a C₆-C₁₀ aryl group, a C₅-C₈ saturated cyclic group, or a 5-10 membered heterocyclic group; 1 or 2 carbon atoms in the foregoing heterocyclic moieties are optionally substituted by an oxo (═O) moiety; and the alkyl, aryl and heterocyclic moieties of the foregoing R⁴ groups are optionally substituted by 1 to 3 substituents independently selected from halo, cyano, nitro, trifluoromethyl, trifluoromethoxy, azido, —NR⁶SO₂R⁵, —SO₂NR⁵R⁶, —C(O)R⁵, —C(O)OR⁵, —OC(O)R⁵, —NR⁶C(O)R⁵, —C(O)NR⁵R⁶, —NR⁵R⁶, —(CR⁶R⁷)ₘOR⁶ wherein m is an integer from 1 to 5, —OR⁵ and the substituents listed in the definition of R⁵;

Each R⁵ is independently selected from H, C₁-C₁₀ alkyl, —(CH₂)ₜ(C₆-C₁₀ aryl), and —(CH₂)ₜ(5-10 membered heterocyclic), wherein t is an integer from 0 to 5; said alkyl group optionally includes 1 or 2 hetero moieties selected from O, S and —N(R⁶)— with the proviso that two O atoms, two S atoms, or an O and S atom are not attached directly to each other; said aryl and heterocyclic R⁵ groups are optionally fused to a C₆-C₁₀ aryl group, a C₅-C₈ saturated cyclic group, or a 5-10 membered heterocyclic group; and the foregoing R⁵ substituents, except H, are optionally substituted by 1 to 3 substituents independently selected from halo, cyano, nitro, trifluoromethyl, trifluoromethoxy, azido, —C(O)R⁶, —C(O)OR⁶, —CO(O)R⁶, —NR⁶C(O)R⁷, —C(O)NR⁶R⁷, —NR⁶R⁷, hydroxy, C₁-C₆ alkyl, and C₁-C₆ alkoxy, and,

each R⁶ and R⁷ is independently H or C₁-C₆ alkyl.

In one embodiment of the method of the present invention the taxane is selected from the group consisting of paclitaxel and docetaxel.

In one preferred embodiment of the method of the present invention the taxane is paclitaxel.

In another preferred embodiment of the method of the present invention, the taxane is docetaxel.

In another preferred embodiment of the method of the present invention the nucleoside analog is Gemzar® (gemcitabine hydrochloride).

In one embodiment of the method of the present invention the platinum coordination complex is selected from the group consisting of cisplatin, carboplatin, tetraplatin and topotecan.

In a preferred embodiment of the present invention the platinum coordination complex is selected from the group consisting of carboplatin and tetraplatin.

In a more preferred embodiment of the present invention the platinum coordination complex is carboplatin.

In another preferred embodiment of the method of the present invention the nucleoside analog is 5-fluorouracil.

In another embodiment of the method of the present invention the anthracycline is selected from the group consisting of doxorubicin, carminomycin and aclacinomycin.

In a preferred embodiment of the present invention the anthracycline is doxorubicin.

In one embodiment of the present invention the topoisomerase inhibitor is selected from the group consisting of etoposide, teniposide, amsacrine, topotecan, and Camptosar® (also known as CPT-11 or irinotecan HCl).

In a preferred embodiment the topoisomerase is Camptosar®.

In another embodiment of the present invention the aromatase inhibitor is selected from the group consisting of letrazole, vorazole, Aromasin® (exemestane) (Pharmacia, Inc., Kalamazoo, Mich.) and anastrazole.

In a preferred embodiment the aromatase inhibitor is selected from the group consisting of Aromasin® (exemestane), and anastrazole.

In one embodiment of the method of the present invention the hyperproliferative disorder is cancer, wherein said cancer is selected from the group consisting of brain, squamous cell, bladder, gastric, pancreatic, breast, head, neck, oesophageal, prostate, colorectal, lung, renal, kidney, ovarian, gynecological and thyroid cancer.

In one preferred embodiment the cancer is selected from the group consisting of prostate, breast, lung, colon and ovarian cancer.

In a more preferred embodiment the cancer is selected from the group consisting of prostate, breast, and lung cancer.

In one preferred embodiment the breast cancer is metastatic breast cancer.

In another preferred embodiment the lung cancer is non-small cell lung cancer (NSCL).

In another embodiment of the method of the present invention the hyperproliferative disorder is non-cancerous.

In one embodiment the non-cancerous hyperproliferative disorder is benign hyperplasia of the skin or prostate.

In one preferred embodiment of the present invention the therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and the therapeutically effective amount of a compound of the formula 1 are administered simultaneously.

In one preferred embodiment of the present invention the therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor and the therapeutically effective amount of a compound of the formula 1 are administered sequentially

The present invention further relates to a method of treating a hyperproliferative disorder in a mammal which comprises administering to said mammal in need of such treatment, either simultaneously or sequentially, (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of the hydrochloride salt of 3-(4-Bromo-2,6-difluoro-benzyloxy)-5-[3-(4-pyrrolidin-1-yl-butyl)-ureido}-isothiazole-4-carboxylic acid amide.

Preferred compounds include those of formula 1 wherein R² is H and R¹ is C₁-C₁₀ alkyl optionally substituted by 1 or 2 substituents independently selected from —NR⁵R⁶, —NR⁵(CR⁶R⁷)ₜOR⁶ and —(CH₂)ₜ(5-10 membered heterocyclic) wherein t is an integer from 0 to 5. Specific preferred R¹ groups include propyl, butyl, pentyl and hexyl optionally substituted by dimethylamino, hydroxy, pyrrolidinyl, morpholino, and ethyl-(2-hydroxy-ethyl)amino.

Other preferred compounds include those of formula 1 wherein R² is H and R¹ is —(CH₂)ₜ(5-10 membered heterocyclic), wherein t is an integer from 0 to 5; said heterocyclic group is optionally fused to a C₆-C₁₀ aryl group, a C₅-C₈ saturated cyclic group, or a 5-10 membered heterocyclic group; and said R¹ group, including the optionally fused portions of said R¹ group, is optionally substituted by 1 or 2 substituents independently selected from C₁-C₄ alkyl, hydroxy and hydroxymethyl. Specific preferred heterocyclic groups of said R¹ group are morpholino, pyrrolidinyl, imidazolyl, piperazinyl, piperidinyl, and 2,5-diaza-bicyclo[2.2.1]hept-2-yl, the t variable of said R¹ group ranges from 2 to 5, and said heterocyclic groups are optionally substituted by hydroxy, hydroxymethyl and methyl.

Other preferred compounds include those of formula 1 wherein R³ is —(CH₂)ₜ(C₆-C₁₀ aryl) wherein t is an integer from 1 to 3 and said R³ group is optionally substituted by 1 to 4 R⁴ groups. Specific preferred R³ groups include benzyl optionally substituted by 1 to 4 substituents independently selected from halo and C₁-C₄ alkyl. More specific preferred R³ groups include benzyl substituted by 1 to 4 substituents independently selected from methyl, fluoro, chloro and bromo.

The present invention also relates to a method of treating a hyperproliferative disorder in a mammal which comprises administering to said mammal in need of such treatment, either simultaneously or sequentially, (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of a compound of the formula 1; and (iii) a therapeutically effective amount of an anti-hypertensive agent.

In one embodiment of the present invention the anti-hypertensive agent is selected from the group consisting of calcium channel blockers, angiotensin converting enzyme inhibitors (ACE inhibitors), angiotensin II receptor antagonists (A-II antagonists), diuretics, beta-adrenergic receptor blockers (β-blockers), vasodilators and alpha-adrenergic receptor blockers (α-blockers).

In a preferred embodiment of the present invention the anti-hypertensive agent is an angiotensin converting enzyme inhibitor (ACE inhibitor).

In one embodiment the ACE inhibitor is accupril (quinapril) (Pfizer, Inc. N.Y.) or accuretic (quinapril and hydrochlorothiazide) (Pfizer, Inc. N.Y.).

In another preferred embodiment of the present invention the anti-hypertensive agent is an alpha-adrenergic receptor blocker (α-blocker).

In one embodiment of the present invention the alpha-adrenergic receptor blocker (α-blocker) is selected from the group consisting of cardura (doxazosin) (Pfizer, Inc. N.Y.) or cardura XL (doxazosin GITS) (Pfizer, Inc. N.Y.).

In another preferred embodiment of the present invention the anti-hypertensive agent is a calcium channel blocker.

In one embodiment the calcium channel blocker is selected from the group consisting of Norvasc (amlodipine) (Pfizer, Inc. N.Y.), procardia (nifedipine) (Pfizer, Inc. N.Y.) and procardia XL (nifedipine GITS) (Pfizer, Inc. N.Y.).

Specific embodiments of the present invention include the following compounds:
5-{3-[3-(4-Methyl-piperazin-1-yl)-propyl]-ureido}-3-(2,3,6-trifluoro-4-methyl-benzyloxy)-isothiazole-4-carboxylic acid amide;
3-(4-Chloro-2,6-difluoro-benzyloxy)-5-(3-{4-[ethyl-(2-hydroxy-ethyl)-amino]-butyl}-ureido)-isothiazole-4-carboxylic acid amide;
3-(2-Fluoro-4-methyl-benzyloxy)-5-{3-[3-(4-methyl-piperazin-1-yl)-propyl]-ureido}-isothiazole-4-carboxylic acid amide;
3-(2,5-Difluoro-4-methyl-benzyloxy)-5-(3-4-[4-(2-hydroxy-ethyl)-piperazin-1-yl]-butyl}-ureido)-isothiazole-4-carboxylic acid amide;
3-(2,5-Difluoro-4-methyl-benzyloxy)-5-[3-(6-dimethylamino-hexyl)-ureido]-isothiazole-4-carboxylic acid amide;
3-(2-Fluoro-4-methyl-benzyloxy)-5-[3-(5-isopropylamino-pentyl)-ureido]-isothiazole-4-carboxylic acid amide;
3-(4-Chloro-2,6-difluoro-benzyloxy)-5-[3-(4-pyrrolidin-1-yl-butyl)-ureido]-isothiazole-4-carboxylic acid amide;
3-(4-Chloro-2,6-difluoro-benzyloxy)-5-{3-[3-(4-methyl-piperazin-1-yl)-propyl]-ureido}-isothiazole-4-carboxylic acid amide;
3-(4-Chloro-2,6-difluoro-benzyloxy)-5-{3-[-(1-methyl-pyrrolidin-2-yl)-ethyl]-ureido}-isothiazole-4-carboxylic acid amide;
3-(2,6-Difluoro-4-methyl-benzyloxy)-5-[3-(4-pyrrolidin-1-yl-butyl)-ureido]-isothiazole-4-carboxylic acid amide;
3-(2,6-Difluoro-4-methyl-benzyloxy)-5-[3-{4-[4-(2-hydroxy-ethyl)-piperazin-1-yl]-butyl}-ureido)-isothiazole-4-carboxylic acid amide;
3-(4-Chloro-2,6-difluoro-benzyloxy)-5-[3-(3-hydroxy-5-pyrrolidin-1-yl)-pentyl)-ureido}-isothiazole-4-carboxylic acid amide;
3-(2,5-Difluoro-4-methyl-benzyloxy)-5-{3-[4-(3,4-dihydroxy-pyrrolidin-1-yl]-butyl]-ureido}-isothiazole-4-carboxylic acid amide;
3-(4-Chloro-2,6-difluoro-benzyloxy)-5-{3-[4-(3,4-dihydroxy-pyrrolidin-1-yl)-butyl]-ureido}-isothiazole-4-carboxylic acid amide;
3-(2,5-Difluoro-4-methyl-benzyloxy)-5-{3-[4-(2-hydroxymethyl-pyrrolidin-1-yl)-butyl]-ureido}-isothiazole-4-carboxylic acid amide;
3-(4-Chloro-2,6-difluoro-benzyloxy)-5-{3-[4-(2-hydroxymethyl-pyrrolidin-1-yl)-butyl]-ureido}-isothiazole-4-carboxylic acid amide;
3-(2,5-Difluoro-4-methyl-benzyloxy)-5-{3-[4-(3-hydroxy-pyrrolidin-1-yl)-butyl]-ureido}-isothiazole-4-carboxylic acid amide;
3-(4-Bromo-2,6-difluoro-benzyloxy)-5-[3-(4-pyrrolidin-1-yl-butyl)-ureido}-isothiazole-4-carboxylic acid amide;
mesylate salt of 3-(4-Bromo-2,6-difluoro-benzyloxy)-5-[3-(4-pyrrolidin-1-yl-butyl)-ureido}-isothiazole-4-carboxylic acid amide;
3-(2,6-Difluoro-4-methyl-benzyloxy)-5-[3-(4-hydroxy-5-piperidin-1-yl-pentyl)-ureido]-isothiazole-4-carboxylic acid amide;
3-(2,5-Difluoro-4-methyl-benzyloxy)-5-{3-[4-(3-hydroxy-5-piperidin-1-yl-pentyl)-ureido]-isothiazole-4-carboxylic acid amide;
3-(4-Chloro-2,6-difluoro-benzyloxy)-5-{3-[4-(2-hydroxymethyl-piperidin-1-yl)-butyl]-ureido}-isothiazole-4-carboxylic acid amide;
3-(2,5-Difluoro-4-methyl-benzyloxy)-5-(3-{4-[ethyl-(2-hydroxy-ethyl)-amino]-butyl}-ureido)-isothiazole-4-carboxylic acid amide;
3-(4-Chloro-2,6-difluoro-benzyloxy)-5-[3-(5-hydroxy-6-piperidin-1-yl)-hexyl)-ureido}-isothiazole-4-carboxylic acid amide;
3-(4-Bromo-2,3,6-trifluoro-benzyloxy)-5-{3-[3-(4-methyl-piperazin-1-yl)-propyl]-ureido}-isothiazole-4-carboxylic acid amide;
3-(2,6-Difluoro-4-methyl-benzyloxy)-5-{3-[3-(4-methyl-piperazin-1-yl-propyl]-ureido}-isothiazole-4-carboxylic acid amide;
hydrochloride salt of 3-(4-Bromo-2,6-difluoro-benzyloxy)-5-[3-(4-pyrrolidin-1-yl-butyl)-ureido}-isothiazole-4-carboxylic acid amide;
3-(2,6-Difluoro-4-methyl-benzyloxy)-5-[3-(3-hydroxy-5-pyrrolidin-1-yl-pentyl)-ureido]-isothiazole-4-carboxylic acid amide;
5-[3-(4-Pyrrolidin-1-yl-butyl)-ureido]-3-(2,3,6-trifluoro-4-methyl-benzyloxy)-isothiazole-4-carboxylic acid amide;
5-[3-(3-Hydroxy-5-pyrrolidin-1-yl-pentyl)-ureido]-3-(2,3,6-trifluoro-4-methyl-benzyloxy)-isothiazole-4-carboxylic acid amide;
3-(4-Chloro-2,6-difluoro-benzyloxy)-5-{3-[3-(5-methyl-2,5-diazabicyclo[2.2.1]hept-2-yl)-propyl]-ureido}-isothiazole-4-carboxylic acid amide;
3-(4-Chloro-2,3,6-trifluoro-benzyloxy)-5-{3-[3-(5-methyl-2,5-diaza-bicyclo[2.2.1]hept-2-yl)propyl]-ureido}-isothiazole-4-carboxylic acid amide;
3-(4-Chloro-2,3,6-trifluoro-benzyloxy)-5-{3-[2-(1-methyl-pyrrolidin-2-yl)-ethyl]-ureido}-isothiazole-4-carboxylic acid amide;
3-(4-Chloro-2,3,6-trifluoro-benzyloxy)-5-[3-(4-pyrrolidin-1-yl-butyl)-ureido]-isothiazole-4-carboxylic acid amide;
3-(4-Chloro-2,3,6-trifluoro-benzyloxy)-5-{3-[4-(2-hydroxymethyl-pyrrolidin-1-yl)-butyl]-ureido}-isothiazole-4-carboxylic acid amide;
5-{3-[2-(1-Methyl-pyrrolidin-2-yl)-ethyl]-ureido}-3-(2,3,6-trifluoro-4-methyl-benzyloxy)-isothiazole-4-carboxylic acid amide;
5-[3-(4-Dimethylamino-butyl)-ureido]-3-(2,3,6-trifluoro-4-methyl-benzyloxy)-isothiazole-4-carboxylic acid amide;
5-[3-(3-Dimethylamino-propyl)-ureido]-3-(2,3,6-trifluoro-4-methyl-benzyloxy)-isothiazole-4-carboxylic acid amide;
5-[3-(3-Hydroxy-5-isopropropylamino-pentyl)-ureido]-3-(2,3,6-trifluoro-4-methyl-benzyloxy)-isothiazole-4-carboxylic acid amide;
5-[3-(3-Isopropylamino-propyl)-ureido]-3-(2,3,6-trifluoro-4-methyl-benzyloxy)-isothiazole-4-carboxylic acid amide;
5-{3-[4-(4-Methyl-piperazin-1-yl)-butyl]-ureido}-3-(2,3,6-trifluoro-4-methyl-benzyloxy)-isothiazole-4-carboxylic acid amide;
5-(3-{4-[4-(2-Hydroxy-ethyl)-piperazin-1-yl]-butyl}-ureido)-3-(2,3,6-trifluoro-4-methyl-benzyloxy)-isothiazole-4-carboxylic acid amide;
5-[3-(3-Pyrrolidin-1-yl-propyl)-ureido]-3-(2,3,6-trifluoro-4-methyl-benzyloxy)-isothiazole-4-carboxylic acid amide;
5-[3-(4-Hydroxy-5-piperidin-1-yl-pentyl)-ureido]-3-(2,3,6-trifluoro-4-methyl-benzyloxy)-isothiazole-4-carboxylic acid amide;
3-(4Chloro-2,6-difluoro-benzyloxy)-5-[3-(4-imidazol-1-yl-butyl)-ureido]-isothiazole-4-carboxylic acid amide;
5-(3-{4-[Ethyl-(2-hydroxy-ethyl)-amino]-butyl}-ureido)-3-(2,3,6-trifluoro-4-methyl-benzyloxy)-isothiazole-4-carboxylic acid amide;
3-(4-Chloro-(2,3,6-trifluoro-benzyloxy)-5-{3-[4-(2-hydroxmethyl-piperidin-1-yl)-butyl]-ureido}-isothiazole-4-carboxylic acid amide;
3-(4-Chloro-2,3,6-trifluoro-benzyloxy)-5-[3-(3-hydroxy-5-pyrrolidin-1-yl-pentyl)-ureido]-isothiazole-4-carboxylic acid amide;
3-(4-Bromo-2,6-difluoro-benzyloxy)-5-{3-[3-(4-methyl-piperazin-1-yl)-propyl]-ureido}-isothiazole-4-carboxylic acid amide;
3-(2,6-Difluoro-4-methyl-benzyloxy)-5-{3-[2-(1-methyl-pyrrolidin-2-yl)-ethyl]-ureido}-isothiazole-4-carboxylic acid amide;
3-(2,6-Difluoro-4-methyl-benzyloxy)-5-[3-(4-dimethylamino-butyl)-ureido}-isothiazole-4-carboxylic acid amide;
3-(2,6-Difluoro-4-methyl-benzyloxy)-5-[3-(3-dimethylamino-propyl)-ureido]-isothiazole-4-carboxylic acid amide;
3-(4-Bromo-2,3,6-trifluoro-benzyloxy)-5-[3-(4-pyrrolidin-1-yl-butyl)ureido]-isothiazole-4-carboxylic acid amide;
3-(4-Chloro-2,3,6-trifluoro-benzyloxy)-5-[3-(4-imidazol-1-yl-butyl)-ureido]-isothiazole-4-carboxylic acid amide;
3-(4-Chloro-2,3,6-difluoro-benzyloxy)-5-(3-{3-[ethyl-(2-hydroxy-ethyl)-amino]-propyl}-ureido)-isothiazole-4-carboxylic acid amide;
3-(4-Chloro-2,3,6-trifluoro-benzyloxy)-5-(3-{3-[ethyl-(2-hydroxy-ethyl)amino]-propyl}-ureido)-isothiazole-4-carboxylic acid amide;
5-[3-(3-Methylamino-propyl)-ureido]-3-(2,3,6-trifluoro-4-methyl-benzyloxy)-isothiazole-4-carboxylic acid amide;
5-[3-(3-Amino-propyl)-3-methyl-ureido]-3-(2,3,6-trifluoro-4-methyl-benzyloxy)-isothiazole-4-carboxylic acid amide;
5-[3-(4-Diethylamino-butyl)-ureido]-3-(2,3,6-trifluoro-4-methyl-benzyloxy)-isothiazole-4-carboxylic acid amide;
3-(2,6-Difluoro-4-methyl-benzyloxy)-5-[3-(3-pyrrolidin-1-yl-propyl)-ureido]-isothiazole-4-carboxylic acid amide;
3-(3-Chloro-2,6-difluoro-4-methyl-benzyloxy)-5-[3-(4-dimethylamino-butyl)-ureido]-isothiazole-4-carboxylic acid amide;
5-(3-{4-[Bis-(2-hydroxy-ethyl)-amino]-butyl}-ureido)-3-(2,6-difluoro-4-methyl-benzyloxy)-isothiazole-4-carboxylic acid amide;

and the pharmaceutically acceptable salts and hydrates of the foregoing compounds.

Preferred embodiments of the present invention include the following compounds:
3-(4-Bromo-2,6-difluoro-benzyloxy)-5-[3-(4-pyrrolidin-1-yl-butyl)-ureido]-isothiazole-4-carboxylic acid amide
mesylate salt of 3-(4-Bromo-2,6-difluoro-benzyloxy)-5-[3-(4-pyrrolidin-1-yl-butyl)-ureido}-isothiazole-4-carboxylic acid amide;
5-{3-[3-(4-Methyl-piperazin-1-yl)-propyl]-ureido}-3-(2,3,6-trifluoro-4-methyl-benzyloxy)-isothiazole-4-carboxylic acid amide;
3-(4-Chloro-2,6-difluoro-benzyloxy)-5-(3-{4-[ethyl-(2-hydroxy-ethyl)-amino]-butyl}-ureido)-isothiazole-4-carboxylic acid amide;
3-(2-Fluoro-4-methyl-benzyloxy)-5-{3-[3-(4-methyl-piperazin-1-yl)-propyl]-ureido}-isothiazole-4-carboxylic acid amide;
3-(2,5-Difluoro-4-methyl-benzyloxy)-5-(3-4-[4-(2-hydroxy-ethyl)-piperazin-1-yl]-butyl}-ureido)-isothiazole-4-carboxylic acid amide;
3-(2,5-Difluoro-4-methyl-benzyloxy)-5-[3-(6-dimethylamino-hexyl)-ureido]-isothiazole-4-carboxylic acid amide;
3-(2-Fluoro-4-methyl-benzyloxy)-5-[3-(5-isopropylamino-pentyl)-ureido]-isothiazole-4-carboxylic acid amide;
hydrochloride salt of 3-(4-Bromo-2,6-difluoro-benzyloxy)-5-[3-(4-pyrrolidin-1-yl-butyl) -ureido}-isothiazole-4-carboxylic acid amide;

and the pharmaceutically acceptable salts, prodrugs and solvates of said compounds.

More preferred embodiments of the present invention include compounds of formula 1 is selected from the group consisting of
3-(4-Bromo-2,6-difluoro-benzyloxy)-5-[3-(4-pyrrolidin-1-yl-butyl)-ureido]-isothiazole-4-carboxylic acid amide
mesylate salt of 3-(4-Bromo-2,6-difluoro-benzyloxy)-5-[3-(4-pyrrolidin-1-yl-butyl)-ureido}-isothiazole-4-carboxylic acid amide;
hydrochloride salt of 3-(4-Bromo-2,6-difluoro-benzyloxy)-5-[3-(4-pyrrolidin-1-yl-butyl)-ureido}-isothiazole-4-carboxylic acid amide;

and the pharmaceutically acceptable salts, prodrugs and solvates of said compounds.

In a more preferred embodiment of the present invention the compound of formula 1 is a hydrochloride salt of 3-(4-Bromo-2,6-difluoro-benzyloxy)-5-[3-(4-pyrrolidin-1-yl-butyl)-ureido}-isothiazole-4-carboxylic acid amide; and the pharmaceutically acceptable salts, prodrugs and solvates of said compound.

The present invention also relates to a pharmaceutical composition for the treatment of a hyperproliferative disorder in a mammal which comprises (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of a compound of the formula 1, in combination with one or more pharmaceutically acceptable carriers or vehicles.

The invention also relates to a pharmaceutical composition for the treatment of a hyperproliferative disorder in a mammal which comprises (i) a therapeutically effective amount of a compound selected from doxorubicin, 5-FU, carboplatin, paclitaxel, gemcitabine hydrochloride, CPT-11 and exemestane and (ii) a therapeutically effective amount of a compound of the formula 1, in combination with one or more pharmaceutically acceptable carriers or vehicles.

The present invention also relates to a pharmaceutical composition for the treatment of a hyperproliferative disorder in a mammal which comprises (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of a compound of the formula 1; and (iii) a therapeutically effective amount of an anti-hypertensive agent, in combination with one or more pharmaceutically acceptable carriers or vehicles.

In one embodiment, said pharmaceutical composition is for the treatment of cancer such as brain, lung, squamous cell, bladder, gastric, pancreatic, breast, head, neck, renal, prostate, colorectal, oesophageal, gynecological (such as ovarian) or thyroid cancer. In another embodiment, said pharmaceutical composition is for the treatment of a non-cancerous hyperproliferative disorder such as benign hyperplasia of the skin (e.g., psoriasis) or prostate (e.g., benign prostatic hypertrophy (BPH)).

In one preferred embodiment the pharmaceutical composition is for the treatment of cancer selected from brain, squamous cell, bladder, gastric, pancreatic, breast, head, neck, oesophageal, prostate, colorectal, lung, renal, kidney, ovarian, gynecological and thyroid cancer. In a more preferred embodiment the pharmaceutical composition is for the treatment of prostate, breast, lung, colon and ovarian cancer. In an even more preferred embodiment the pharmaceutical composition is for the treatment of prostate, breast, and lung cancer. In a most preferred embodiment the pharmaceutical composition is for the treatment of metastatic breast cancer or NSCL.

The invention also relates to a pharmaceutical composition for the treatment of pancreatitis or kidney disease (including proliferative glomerulonephritis and diabetes-induced renal disease) in a mammal which comprises (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of a compound of the formula 1, in combination with one or more pharmaceutically acceptable carriers or vehicles.

The invention also relates to a pharmaceutical composition for the prevention of blastocyte implantation in a mammal which comprises (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of a compound of the formula 1, in combination with one or more pharmaceutically acceptable carriers or vehicles.

The invention also relates to a pharmaceutical composition for treating a disease related to vasculogenesis or angiogenesis in a mammal which comprises (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of a compound of the formula 1, in combination with one or more pharmaceutically acceptable carriers or vehicles.

In one embodiment, said pharmaceutical composition is for treating a disease selected from the group consisting of tumor angiogenesis, chronic inflammatory disease such as rheumatoid arthritis, atherosclerosis, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

In one embodiment, the method of the present invention relates to the treatment of cancer such as brain, squamous cell, bladder, gastric, pancreatic, breast, head, neck, oesophageal, prostate, colorectal, lung, renal, gynecological (such as ovarian) or thyroid cancer. In another embodiment, said method relates to the treatment of a non-cancerous hyperproliferative disorder such as benign hyperplasia of the skin (e.g., psoriasis) or prostate (e.g., benign prostatic hypertrophy (BPH)).

The invention also relates to a method of treating pancreatitis or kidney disease in a mammal which comprises administering to said mammal, either simultaneously or sequentially, (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of a compound of the formula 1, in combination with one or more pharmaceutically acceptable carriers or vehicles.

The invention also relates to a method of preventing blastocyte implantation in a mammal which comprises administering to said mammal, either simultaneously or sequentially, (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of a compound of the formula 1.

The invention also relates to a method of treating diseases related to vasculogenesis or angiogenesis in a mammal which comprises administering to said mammal, either simultaneously or sequentially, (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of a compound of the formula 1.

In one embodiment, said method is for treating a disease selected from the group consisting of tumor angiogenesis, chronic inflammatory disease such as rheumatoid arthritis, atherosclerosis, skin diseases such as psoriasis, eczema, and scleroderma, diabetes, diabetic retinopathy, retinopathy of prematurity, macular degeneration, hemangioma, glioma, melanoma, Kaposi's sarcoma and ovarian, breast, lung, pancreatic, prostate, colon and epidermoid cancer.

Patients that can be treated with compounds of formulas 1 and the pharmaceutically acceptable salts and hydrates of said compounds, in combination with a taxane derivative, a platinum coordination complex, a nucleoside analog, an anthracycline, a topoisomerase inhibitor, or an aromatase inhibitor, according to the methods of this invention include, for example, patients that have been diagnosed as having psoriasis, BPH, lung cancer, bone cancer, pancreatic cancer, skin cancer, cancer of the head and neck, cutaneous or intraocular melanoma, uterine cancer, ovarian cancer, rectal cancer or cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas).

The present invention also relates to a kit comprising in a first compartment a compound of formula 1 and in a second compartment a taxane derivative, a platinum coordination complex, a nucleoside analog, an anthracycline, a topoisomerase inhibitor, or an aromatase inhibitor.

The present invention also relates to a kit comprising in a first compartment a compound of formula 1, a second compartment a taxane derivative, a platinum coordination complex, a nucleoside analog, an anthracycline, a topoisomerase inhibitor, or an aromatase inhibitor, and a third compartment containing an anti-hypertensive agent.

In one embodiment of the kit of the present invention the compound in the second compartment is 5-FU.

In another embodiment of the kit of the present invention the second compartment is carboplatin.

In another preferred embodiment of the kit of the present invention the compound in the second compartment is doxorubicin.

In one preferred embodiment of the kit of the present invention the compound in the second compartment is paclitaxel.

In another preferred embodiment of the kit of the present invention the second compartment is gemcitabine hydrochloride.

In another preferred embodiment of the kit of the present invention the second compartment is CPT-11.

In another preferred embodiment of the kit of the present invention the second compartment is exemestane.

The terms “concurrently” and “simultaneously” are used interchangeably and mean the compounds of the combination therapy of the present invention are administered (1) simultaneously in time, or (2) at different times during the course of a common treatment schedule.

The term “sequentially” as used herein means (1) administration of one component of the method ((i) a compound of formula 1 or (ii) a taxane derivative, a platinum coordination complex, a nucleoside analog, an anthracycline, a topoisomerase inhibitor, or an aromatase inhibitor) followed by administration of the other component; after administration of one component, the second component can be administered substantially immediately after the first component, or the second component can be administered after an effective time period after the first component; the effective time period is the amount of time given for realization of maximum benefit from the administration of the first component.

The term “halo”, as used herein, unless otherwise indicated, includes fluoro, chloro, bromo or iodo. Preferred halo groups are fluoro, chloro and bromo.

The term “alkyl”, as used herein, unless otherwise indicated, includes saturated monovalent hydrocarbon radicals having straight, cyclic or branched moieties. It is understood that for cyclic moieties at least three carbon atoms are required in said alkyl group.

The term “alkenyl”, as used herein, unless otherwise indicated, includes monovalent hydrocarbon radicals having at least one carbon-carbon double bond and also having straight, cyclic or branched moieties as provided above in the definition of “alkyl”.

The term “alkynyl”, as used herein, unless otherwise indicated, includes monovalent hydrocarbon radicals having at least one carbon-carbon triple bond and also having straight, cyclic or branched moieties as provided above in the definition of “alkyl”.

The term “alkoxy”, as used herein, unless otherwise indicated, includes O-alkyl groups wherein “alkyl” is as defined above.

The term “aryl”, as used herein, unless otherwise indicated, includes an organic radical derived from an aromatic hydrocarbon by removal of one hydrogen, such as phenyl or naphthyl.

The term “4-10 membered heterocyclic”, as used herein, unless otherwise indicated, includes aromatic and non-aromatic heterocyclic groups containing one or more heteroatoms each selected from O, S and N, wherein each heterocyclic group has from 4-10 atoms in its ring system. Non-aromatic heterocyclic groups include groups having only 4 atoms in their ring system, but aromatic heterocyclic groups must have at least 5 atoms in their ring system. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, tetrahydrothienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the compounds listed above, may be C-attached or N-attached where such is possible. For instance, a group derived from pyrrole may be pyrrol-1-yl (N-attached) or pyrrol-3-yl (C-attached).

The phrase “pharmaceutically acceptable salt(s)”, as used herein, unless otherwise indicated, includes salts of acidic or basic groups which may be present in the compounds of formula 1. The compounds of formula 1 that are basic in nature are capable of forming a wide variety of salts with various inorganic and organic acids. The acids that may be used to prepare pharmaceutically acceptable acid addition salts of such basic compounds of formula 1 are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts.

Those compounds of the formula 1 that are acidic in nature, are capable of forming base salts with various pharmacologically acceptable cations. Examples of such salts include the alkali metal or alkaline earth metal salts and particularly, the sodium and potassium salts.

Certain compounds of formula 1 may have asymmetric centers and therefore exist in different enantiomeric forms. This invention relates to the use of all optical isomers and stereoisomers of the compounds of formula 1 and mixtures thereof. The compounds of formula 1 may also exist as tautomers. This invention relates to the use of all such tautomers and mixtures thereof.

The subject invention also includes isotopically-labelled compounds, and the pharmaceutically acceptable salts thereof, which are identical to those recited in formula 1, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula 1 of this invention and prodrugs thereof can generally be prepared by carrying out the procedures disclosed in the Schemes and/or in the Examples and Preparations below, by substituting a readily available isotopically labelled reagent for a non-isotopically labelled reagent.

Compounds of formula 1 having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula 1. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs are also encompassed. For instance, free carboxyl groups can be derivatized as amides or alkyl esters. The amide and ester moieties may incorporate groups including but not limited to ether, amine and carboxylic acid functionalities. Free hydroxy groups may be derivatized using groups including but not limited to hemisuccinates, phosphate esters, dimethylaminoacetates, and phosphoryloxymethyloxycarbonyls, as outlined in D. Fleisher, R. Bong, B. H. Stewart, Advanced Drug Delivery Reviews (1996) 19, 115. Carbamate prodrugs of hydroxy and amino groups are also included, as are carbonate prodrugs and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in R. P. Robinson et al., J. Medicinal Chemistry (1996) 39, 10.

### DETAILED DESCRIPTION OF THE INVENTION

The present invention relates to a method of treating a hyperproliferative disorder in a mammal which comprises administering to said mammal in need of such treatment, either simultaneously (concurrently) or sequentially, (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor, and (ii) a therapeutically effective amount of a compound of the formula 1

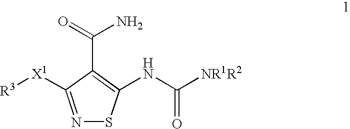

or a pharmaceutically acceptable salt, prodrug or solvate thereof, wherein:

wherein X¹ is O or S;

R¹ is H, C₁-C₁₀ alkyl, C₂-C₁₀ alkenyl, C₂-C₁₀ alkynyl, —C(O)(C₁-C₁₀ alkyl), —(CH₂)ₜ(C₆-C₁₀ aryl), —(CH₂)ₜ(4-10 membered heterocyclic), —C(O)(CH₂)ₜ(C₆-C₁₀ aryl), or —C(O)(CH₂)ₜ(5-10 membered heterocyclic), wherein t is an integer from 0 to 5; said alkyl group optionally includes 1 or 2 hetero moieties selected from O, S and —N(R⁶)— with the proviso that two O atoms, two S atoms, or an O and S atom are not attached directly to each other; said aryl and heterocyclic R¹ groups are optionally fused to a C₆-C₁₀ aryl group, a C₅-C₈ saturated cyclic group, or a 5-10 membered heterocyclic group; 1 or 2 carbon atoms in the foregoing heterocyclic moieties are optionally substituted by an oxo (═O) moiety; the —(CH₂)ₜ— moieties of the foregoing R¹ groups optionally include a carbon-carbon double or triple bond where t is an integer from 2 to 5; and the foregoing R¹ groups, except H, are optionally substituted by 1 to 3 R⁴ groups;

R² is selected from the list of substituents provided in the definition of R¹, —SO₂(CH₂)ₜ(C₆-C₁₀ aryl), —SO₂(CH₂)ₜ(5-10 membered heterocyclic), and —OR⁵, t is an integer ranging from 0 to 5, the —(CH₂)ₜ— moieties of the foregoing R² groups optionally include a carbon-carbon double or triple bond where t is an integer from 2 to 5, and the foregoing R² groups are optionally substituted by 1 to 3 R⁴ groups;

or R¹ and R² may be taken together with the nitrogen to which each is attached to form a 4-10 membered saturated monocyclic or polycyclic ring or a 5-10 membered heteroaryl ring, wherein said saturated and heteroaryl rings optionally include 1 or 2 heteroatoms selected from O, S and —N(R⁶)— in addition to the nitrogen to which R¹ and R² are attached, said —N(R⁶)— is optionally ═N— or —N═ where R¹ and R² are taken together as said heteroaryl group, said saturated ring optionally may be partially unsaturated by including 1 or 2 carbon-carbon double bonds, and said saturated and heteroaryl rings, including the R⁶ group of said —N(R⁶)—, are optionally substituted by 1 to 3 R⁴ groups;

R³ is H, C₁-C₁₀ alkyl, C₂-C₁₀ alkenyl, C₂-C₁₀ alkynyl, —(CH₂)ₜ(C₆-C₁₀ aryl), or —(CH₂)ₜ(5-10 membered heterocyclic), wherein t is an integer from 0 to 5; said alkyl group optionally includes 1 or 2 hetero moieties selected from O, S and —N(R⁶)— with the proviso that two O atoms, two S atoms, or an O and S atom are not attached directly to each other; said aryl and heterocyclic R³ groups are optionally fused to a C₆-C₁₀ aryl group, a C₅-C₈ saturated cyclic group, or a 5-10 membered heterocyclic group; 1 or 2 carbon atoms in the foregoing heterocyclic moieties are optionally substituted by an oxo (═O) moiety, the —(CH₂)ₜ— moieties of the foregoing R³ groups optionally include a carbon-carbon double or triple bond where t is an integer from 2 to 5, and the foregoing R³ groups are optionally substituted by 1 to 5 R⁴ groups;

each R⁴ is independently selected from C₁-C₁₀ alkyl, C₂-C₁₀ alkenyl, C₂-C₁₀ alkynyl, halo, cyano, nitro, trifluoromethyl, trifluoromethoxy, azido, —OR⁵, —C(O)R⁵, —C(O)OR⁵, —NR⁶C(O)OR⁵, —OC(O)R⁵, —NR⁶SO₂R⁵, —SO₂NR⁵R⁶, —NR⁶C(O)R⁵, —C(O)NR⁵R⁶, —NR⁵R⁶, —S(O)ⱼR⁷ wherein j is an integer ranging from 0 to 2, —SO₃H, —NR⁵(CR⁶R⁷)ₜOR⁶, —(CH₂)ₜ(C₆-C₁₀ aryl), —SO₂(CH₂)ₜ(C₆-C₁₀ aryl), —S(CH₂)ₜ(C₆-C₁₀ aryl), —O(CH₂)ₜ(C₆-C₁₀ aryl), —(CH₂)ₜ(5-10 membered heterocyclic), and —(CR⁶R⁷)ₘOR⁶, wherein m is an integer from 1 to 5 and t is an integer from 0 to 5; said alkyl group optionally contains 1 or 2 hetero moieties selected from O, S and —N(R⁶)— with the proviso that two O atoms, two S atoms, or an O and S atom are not attached directly to each other; said aryl and heterocyclic R⁴ groups are optionally fused to a C₆-C₁₀ aryl group, a C₅-C₈ saturated cyclic group, or a 5-10 membered heterocyclic group; 1 or 2 carbon atoms in the foregoing heterocyclic moieties are optionally substituted by an oxo (═O) moiety; and the alkyl, aryl and heterocyclic moieties of the foregoing R⁴ groups are optionally substituted by 1 to 3 substituents independently selected from halo, cyano, nitro, trifluoromethyl, trifluoromethoxy, azido, —NR⁶SO₂R⁵, —SO₂NR⁵R⁶, —C(O)R⁵, —C(O)OR⁵, —OC(O)R⁵, —NR⁶C(O)R⁵, —C(O)NR⁵R⁶, —NR⁵R⁶, —(CR⁶R⁷)ₘOR⁶ wherein m is an integer from 1 to 5, —OR⁵ and the substituents listed in the definition of R⁵;

each R⁵ is independently selected from H, C₁-C₁₀ alkyl, —(CH₂)ₜ(C₆-C₁₀ aryl), and —(CH₂)ₜ(5-10 membered heterocyclic), wherein t is an integer from 0 to 5; said alkyl group optionally includes 1 or 2 hetero moieties selected from O, S and —N(R⁶)— with the proviso that two O atoms, two S atoms, or an O and S atom are not attached directly to each other; said aryl and heterocyclic R⁵ groups are optionally fused to a C₆-C₁₀ aryl group, a C₅-C₈ saturated cyclic group, or a 5-10 membered heterocyclic group; and the foregoing R⁵ substituents, except H, are optionally substituted by 1 to 3 substituents independently selected from halo, cyano, nitro, trifluoromethyl, trifluoromethoxy, azido, —C(O)R⁶, —C(O)OR⁶, —CO(O)R⁶, —NR⁶C(O)R⁷, —C(O)NR⁶R⁷, —NR⁶R⁷, hydroxy, C₁-C₆ alkyl, and C₁-C₆ alkoxy; and,

each R⁶ and R⁷ is independently H or C₁-C₆ alkyl.

Compounds of the formula 1 and their pharmaceutically acceptable salts and solvates may be prepared as described in U.S. Pat. No. 6,235,764, the contents of which are incorporated by reference.

One element of the combination therapy of the present invention includes a taxane derivative. The taxanes are a family of terpenes, including, but not limited to paclitaxel (Taxol®) and docetaxel (Taxotere®), which were derived primarily from the Pacific yew tree, Taxus brevifolia, and which have activity against certain tumors, particularly breast and ovarian tumors. Paclitaxel is a preferred taxane and is considered an antimicrotubule agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions.

The term “paclitaxel” herein includes both naturally derived and related forms and chemically synthesized compounds or derivatives thereof with antineoplastic properties including deoxygenated paclitaxel compounds such as those described in U.S. Pat. No. 5,440,056, herein incorporated by reference, and that sold as Taxol® by Bristol-Myers Oncology. In addition to Taxol®, other derivatives are mentioned in “Synthesis and Anticancer Activity of Taxol other Derivatives,” D. G. I. Kingston et al., Studies in Organic Chemistry, vol. 26, entitled “New Trends in Natural Products Chemistry” (1986), Atta-ur-Rabman, P. W. le Quesne, Eds. (Elvesier, Amsterdam 1986), pp 219-235 are explicitly included here.

In another embodiment of the present invention comprises a platinum coordination complex. Generally, the platinum containing anti-neoplastic agent may be any platinum coordination complex that has an anti-neoplastic effect. More preferably, the platinum containing anti-neoplastic agent of the composition of the present invention is cisplatin or carboplatin (cis-diammine-1,1-cyclobutanedicarboxylato-platinum II), CBDCA, JM-8 and NSC 241240) but could include tetraplatin and topotecan.

In a most preferred embodiment of the present invention the platinum coordination complex is carboplatin. Carboplatin is available commercially as Paraplatin® (Bristol-Myers Squibb, N.J.). The product is supplied as a crystalline white powder in vials containing 50, 150, and 450 mg, and the powder is reconstituted with either Sterile Water for Injection, 5% Dextrose in Water, or 0.9% Sodium Chloride for Injection.

Anthracyclines of the daunorubicin group such as doxorubicin, carminomycin and aclacinomycin and their synthetic analogs are among the most widely employed agents in antitumoral therapy (F. Arcamone, Doxorubicin, Academic Press New York, 1981, pp. 12-25; A. Grein, Process Biochem., 16:34, 1981; T. Kaneko, Chimicaoggi May 11, 1988; C. E. Myers et al., “Biochemical mechanism of tumor cell kill” in Anthracycline and Anthracenedione-Based Anti-cancer Agents (Lown, J. W., ed.) Elsevier Amsterdam, pp. 527-569, 1988; J. W. Lown, Pharmac. Ther. 60:185-214, 1993. Anthracyclines of the daunorubicin group are naturally occurring compounds produced by various Streptomyces species and by Actinomyces carminata. Doxorubicin is mainly produced by strains of Streptomyces peucetius while daunorubicin is produced by many other Actinomycetes. In particular daunorubicin and doxorubicin are synthesized in S. peucetius ATCC 29050 and 27952 from malonic acid, propionic acid and glucose by the pathway summarized in Grein (Advan. Applied Microbiol. 32:203, 1987) and in Eckart and Wagner (J. Basic Microbiol. 28:137, 1988). Doxorubicin is a drug of choice in the clinical management of breast cancer.

In a preferred embodiment of the present invention doxorubicin is used in combination with a compound of formula 1.

Anti-metabolite nucleosides and nucleoside analogs have found widespread use in the treatment of cancer and other human diseases. One such nucleoside analog, 5-fluorouracil (5-FU) has been used continuously since its development in 1957 by Duusinski and Heidelberger (U.S. Pat. No. 2,802,005) for the treatment of solid tumors of the head and neck, breast, and colon. 5-FU was originally designed to work as an inhibitor of thymidylate synthetase (TS), the enzyme which converts deoxyuridine 5′-O-monophosphate (dUMP) to deoxythymidine 5′-O-monophosphate (dTMP). It is believed that 5-FU retards tumor expansion by causing thymidine pools to become depleted in rapidly proliferating tumor cells. Other nucleoside analogs such as gemcitabine hydrochloride are known and are a preferred compound for use in the methods and pharmaceutical compositions of the present invention.

Aromatase inhibiting agents have been found to be particularly useful in the treatment of estrogen dependent disease, e.g., breast cancer or benign prostatic hyperplasia (BPH). It has been reported in the literature that estrogen synthesis can be blocked specifically by inhibiting the enzyme aromatase, which is the key enzyme in the biochemical estrogen synthesis pathway. Aromatase inhibition is important because several breast tumors synthesize estradiol and estrone in situ and therefore exhibit continuous growth stimulation (Alan Lipton et al., Cancer, 59, 770-782, 1987). Aromatase inhibiting agents include the following: letrazole, vorazole, Aromasin® (exemestane), and anastrazole.

Topoisomerases are known as enzymes which temporarily break one strand of the DNA double helix (topoisomerase I or “topo I”) or which simultaneously break two strands of the DNA double helix (“topo II”) in order to effect changes in the topological form of the DNA. Known topoisomerase inhibitors include etoposide, teniposide, amsacrine, topotecan, and Camptosar®.

The present invention also relates to a method of treating a hyperproliferative disorder in a mammal which comprises administering to said mammal in need of such treatment, either simultaneously (concurrently) or sequentially, (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of a compound of the formula 1; and (iii) a therapeutically effective amount of an anti-hypertensive agent. When combinations of the present invention are administered sequentially each agent (i)-(iii) may be administered, first, second or third. In one preferred embodiment, the agents of the combination are administered as agent (i), the taxane derivative, platinum coordination complex, nucleoside analog, or anthracycline, followed by agent (ii) a compound of formula 1; and then agent (iii), the anti-hypertensive agent.

According to a further feature of the invention there is provided a pharmaceutical composition comprising a combination of (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of a compound of the formula 1; and (iii) a therapeutically effective amount of an anti-hypertensive agent for the treatment of a disease state associated with angiogenesis in a warm-blooded mammal, such as a human being.

Combinations of the invention may be administered sequentially or may be administered simultaneously.

The term “anti-hypertensive” means any agent, which lowers blood pressure. There are many different categories of anti-hypertensive agents including calcium channel blockers, angiotensin converting enzyme inhibitors (ACE inhibitors), angiotensin II receptor antagonists (A-II antagonists), diuretics, beta-adrenergic receptor blockers (β-blockers), vasodilators and alpha-adrenergic receptor blockers (α-blockers). Any anti-hypertensive agent may be used in accordance with this invention and examples from each class are given hereinafter.

Calcium channel blockers, which are within the scope of this invention include, but are not limited to: Norvasc (amlodipine) (U.S. Pat. No. 4,572,909); procardia (nifedipine) (Pfizer, Inc. N.Y.); procardia XL (nifedipine GITS) (Pfizer, Inc. N.Y.); bepridil (U.S. Pat. No. 3,962,238 or U.S. Reissue No. 30,577); clentiazem (U.S. Pat. No. 4,567,175); diltiazem (U.S. Pat. No. 3,562,257); fendiline (U.S. Pat. No. 3,262,977); gallopamil (U.S. Pat. No. 3,261,859); mibefradil (U.S. Pat. No. 4,808,605); prenylamine (U.S. Pat. No. 3,152,173); semotiadil (U.S. Pat. No. 4,786,635); terodiline (U.S. Pat. No. 3,371,014); verapamil (U.S. Pat. No. 3,261,859); aranidipine (U.S. Pat. No. 4,446,325); bamidipine (U.S. Pat. No. 4,220,649); benidipine (European Patent Application Publication No. 106,275); cilnidipine (U.S. Pat. No. 4,672,068); efonidipine (U.S. Pat. No. 4,885,284); elgodipine (U.S. Pat. No. 4,952,592); felodipine (U.S. Pat. No. 4,264,611); isradipine (U.S. Pat. No. 4,466,972); lacidipine (U.S. Pat. No. 4,801,599); lercanidipine (U.S. Pat. No. 4,705,797); manidipine (U.S. Pat. No. 4,892,875); nicardipine (U.S. Pat. No. 3,985,758); nifedipine (U.S. Pat. No. 3,485,847); nilvadipine (U.S. Pat. No. 4,338,322); nimodipine (U.S. Pat. No. 3,799,934); nisoldipine (U.S. Pat. No. 4,154,839); nitrendipine (U.S. Pat. No. 3,799,934); cinnarizine (U.S. Pat. No. 2,882,271); flunarizine (U.S. Pat. No. 3,773,939); lidoflazine (U.S. Pat. No. 3,267,104); lomerizine (U.S. Pat. No. 4,663,325); bencyclane (Hungarian Patent No. 151,865); etafenone (German Patent No. 1,265,758); and perhexiline (British Patent No. 1,025,578). The disclosures of all such patents and patent applications are incorporated herein by reference.

Angiotensin Converting Enzyme Inhibitors (ACE-Inhibitors) which are within the scope of this invention include, but are not limited to: accupril (quinapril) (Pfizer, Inc. N.Y.); accuretic (quinapril and hydrochlorothiazide) (Pfizer, Inc. N.Y.); alacepril (U.S. Pat. No. 4,248,883); benazepril (U.S. Pat. No. 4,410,520); captopril (U.S. Pat. Nos. 4,046,889 and 4,105,776); ceronapril (U.S. Pat. No. 4,452,790); delapril (U.S. Pat. No. 4,385,051); enalapril (U.S. Pat. No. 4,374,829); fosinopril (U.S. Pat. No. 4,37,201); imidapril (U.S. Pat. No. 4,508,727); lisinopril (U.S. Pat. No. 4,555,502); moveltipril (Belgium Patent No. 893,553); perindopril (U.S. Pat. No. 4,508,729); quinapril (U.S. Pat. No. 4,344,949); ramipril (U.S. Pat. No. 4,587,258); spirapril (U.S. Pat. No. 4,470,972); temocapril (U.S. Pat. No. 4,699,905); and trandolapril (U.S. Pat. No. 4,933,361). The disclosures of all such patents are incorporated herein by reference.

Angiotensin-II receptor antagonists (A-II antagonists) which are within the scope of this invention include, but are not limited to: candesartan (U.S. Pat. No. 5,196,444); eprosartan (U.S. Pat. No. 5,185,351); irbesartan (U.S. Pat. No. 5,270,317); losartan (U.S. Pat. No. 5,138,069); and valsartan (U.S. Pat. No. 5,399,578). The disclosures of all such U.S. patents are incorporated herein by reference.

β-Blockers which are within the scope of this invention include, but are not limited to: acebutolol (U.S. Pat. No. 3,857,952); alprenolol (Netherlands Patent Application No. 6,605,692); amosulalol (U.S. Pat. No. 4,217,305); arotinolol (U.S. Pat. No. 3,932,400); atenolol (U.S. Pat. Nos. 3,663,607 and 3,836,671); befunolol (U.S. Pat. No. 3,853,923); betaxolol (U.S. Pat. No. 4,252,984); bevantolol (U.S. Pat. No. 3,857,891); bisoprolol (U.S. Pat. No. 4,258,062); bopindolol (U.S. Pat. No. 4,340,541); bucumolol (U.S. Pat. No. 3,663,570); bufetolol (U.S. Pat. No. 3,723,476); bufuralol (U.S. Pat. No. 3,929,836); bunitrolol (U.S. Pat. No. 3,541,130); bupranolol (U.S. Pat. No. 3,309,406); butidrine hydrochloride (French Patent No. 1,390,056); butofilolol (U.S. Pat. No. 4,302,601); carazolol (German Patent No. 2,240,599); carteolol (U.S. Pat. No. 3,910,924); carvedilol (U.S. Pat. No. 4,503,067); celiprolol (U.S. Pat. No. 4,034,009); cetamolol (U.S. Pat. No. 4,059,622); cloranolol (German Patent No. 2, 213,044); dilevalol (Clifton et al., Journal of Medicinal Chemistry, 1982, 25, 670); epanolol (U.S. Pat. No. 4,167,581); indenolol (U.S. Pat. No. 4,045,482); labetalol (U.S. Pat. No. 4,012,444); levobunolol (U.S. Pat. No. 4,463,176); mepindolol (Seeman et al, Helv. Chim. Acta, 1971, 54, 2411); metipranolol (Czechoslovakian Patent Application No. 128,471); metoprolol (U.S. Pat. No. 3,873,600); moprolol (U.S. Pat. No. 3,501,769); nadolol (U.S. Pat. No. 3,935,267); nadoxolol (U.S. Pat. No. 3,819,702); nebivalol (U.S. Pat. No. 4,654,362); nipradilol (U.S. Pat. No. 4,394,382); oxprenolol (British Patent No. 1,077,603); penbutolol (U.S. Pat. No. 3,551,493); pindolol (Swiss Patents Nos. 469,002 and 472,404); practolol (U.S. Pat. No. 3,408,387); pronethalol (British Patent No. 909,357); propranolol (U.S. Pat. Nos. 3,337,628 and 3,520,919); sotalol (Uloth et al., Journal of Medicinal Chemistry, 1966, 9, 88); sulfinalol (German Patent No. 2,728,641); talinolol (U.S. Pat. Nos. 3,935,259 and 4,038,313); tertatolol (U.S. Pat. No. 3,960,891); tilisolol (U.S. Pat. No. 4,129,565); timolol (U.S. Pat. No. 3,655,663); toliprolol (U.S. Pat. No. 3,432,545); and xibenolol (U.S. Pat. No. 4,018,824. The disclosures of all such patents, patent applications and references are incorporated herein by reference.

α-Blockers which are within the scope of this invention include, but are not limited to: cardura (doxazosin) (Pfizer, Inc. N.Y.); cardura XL (doxazosin GITS) (Pfizer, Inc. N.Y.); amosulalol (U.S. Pat. No. 4,217,305); arotinolol; dapiprazole (U.S. Pat. No. 4,252,721); doxazosin (U.S. Pat. No. 4,188,390); fenspiride (U.S. Pat. No. 3,399,192); indoramin (U.S. Pat. No. 3,527,761); labetolol, naftopidil (U.S. Pat. No. 3,997,666); nicergoline (U.S. Pat. No. 3,228,943); prazosin (U.S. Pat. No. 3,511,836); tainsulosin (U.S. Pat. No. 4,703,063); tolazoline (U.S. Pat. No. 2,161,938); trimazosin (U.S. Pat. No. 3,669,968); and yohimbine, which may be isolated from natural sources according to methods well known to those skilled in the art. The disclosures of all such U.S. patents are incorporated herein by reference.

The term “vasodilator”, where used herein, is meant to include cerebral vasodilators, coronary vasodilators and peripheral vasodilators. Cerebral vasodilators within the scope of this invention include, but are not limited to: bencyclane; cinnarizine; citicoline, which may be isolated from natural sources as disclosed in Kennedy et al., Journal of the American Chemical Society, 1955, 77, 250 or synthesised as disclosed in Kennedy, Journal of Biological Chemistry, 1956, 222, 185; cyclandelate (U.S. Pat. No. 3,663,597); ciclonicate (German Patent No. 1,910,481); diisopropylamine dichloroacetate (British Patent No. 862,248); eburnamonine (Hermann et al., Journal of the American Chemical Society, 1979, 101, 1540); fasudil (U.S. Pat. No. 4,678,783); fenoxedil (U.S. Pat. No. 3,818,021); flunarizine (U.S. Pat. No. 3,773,939); ibudilast (U.S. Pat. No. 3,850,941); ifenprodil (U.S. Pat. No. 3,509,164); lomerizine (U.S. Pat. No. 4,663,325); nafronyl (U.S. Pat. No. 3,334,096); nicametate (Blicke et al., Journal of the American Chemical Society, 1942, 64, 1722); nicergoline; nimodipine (U.S. Pat. No. 3,799,934); papaverine, which may be prepared as reviewed in Goldberg, Chem. Prod. Chem. News, 1954, 17, 371; pentifylline (German Patent No. 860,217); tinofedrine (U.S. Pat. No. 3,767,675); vincamine (U.S. Pat. No. 3,770,724); vinpocetine (U.S. Pat. No. 4,035,750); and viquidil (U.S. Pat. No. 2,500,444). The disclosures of all such patents and references are incorporated herein by reference.

Coronary vasodilators within the scope of this invention include, but are not limited to: amotriphene (U.S. Pat. No. 3,010,965); bendazol (Feitelson, et al., I Chem. Soc. 195, 8, 2426); benfurodil hemisuccinate (U.S. Pat. No. 3,355,463); benziodarone (U.S. Pat. No. 3,012,042); chloracizine (British Patent No. 740,932) chromonar (U.S. Pat. No. 3,282,938); clobenfural (British Patent No. 1,160,925); clonitrate, which may be prepared from propanediol according to methods well known to those skilled in the art, e.g., see Annalen, 1870, 155, 165; cloricromen (U.S. Pat. No. 4,452,811); dilazep (U.S. Pat. No. 3,532,685); dipyridamole (British Patent No. 807,826); droprenilamine (German Patent No. 2,521,113); efloxate (British Patents Nos. 803,372 and 824,547); erythrityl tetranitrate, which may be prepared by nitration of erythritol according to methods well-known to those skilled in the art, etafenone (German Patent No. 1,265,758); fendiline (U.S. Pat. No. 3,262,977); floredil (German Patent No. 2,020,464); ganglefene (U.S.S.R. Patent No. 115,905); hexestrol bis(P-diethylaminoethyl) ether (Lowe et al., J. Chem. Soc. 1951, 3286); hexobendine (U.S. Pat. No. 3,267,103) itramin tosylate (Swedish Patent No. 168,308); khellin (Baxter et al., Journal of the Chemical Society, 1949, S30); lidoflazine (U.S. Pat. No. 3,267,104); mannitol hexanitrate, which may be prepared by the nitration of mannitol according to methods well-known to those skilled in the art; medibazine (U.S. Pat. No. 3,119,826); nitroglycerin; pentaerythritol tetranitrate, which may be prepared by the nitration of pentaerythritol according to methods well-known to those skilled in the art; pentrinitrol (German Patent No. 638,422-3); perhexiline; pimefylline (U.S. Pat. No. 3,350,400); prenylamine (U.S. Pat. No. 3,152,173); propatyl nitrate (French Patent No. 1,103,113); trapidil (East German Patent No. 55,956); tricromyl (U.S. Pat. No. 2,769,015); trimetazidine (U.S. Pat. No. 3,262,852); trolnitrate phosphate, which may be prepared by nitration of triethanolamine followed by precipitation with phosphoric acid according to methods well known to those skilled in the art, visnadine (U.S. Pat. Nos. 2,816,118 and 2,980,699). The disclosures of all such patents and references are incorporated herein by reference.

Peripheral vasodilators within the scope of this invention include, but are not limited to: aluminium nicotinate (U.S. Pat. No. 2,970,082); bamethan (Corrigan et al., Journal of the American Chemical Society, 1945, 67, 1894); bencyclane (which may be prepared as described herein before); betahistine (Walter et al, Journal of the American Chemical Society, 1941, 63, 2771); bradykinin (Hamburg et al., Arch. Biochem. Biophys., 1958, 76, 252); brovincamine (U.S. Pat. No. 4,146,643); bufeniode (U.S. Pat. No. 3,542,870); buflomedil (U.S. Pat. No. 3,895,030); butalamine (U.S. Pat. No. 3,38,899); cetiedil (French Patent No. 1,460,571); ciclonicate (German Patent No. 1,910,481); cinepazide (Beiguim Patent No. 730,345); cinnarizine; cyclandelate; diisopropylamine dichloroacetate; eledoisin (British Patent No. 984,8 10); fenoxedil; flunarizine; hepronicate (U.S. Pat. No. 3,384,642); ifenprodil; iloprost (U.S. Pat. No. 4,692,464); inositol niacinate (Badgett et al., Journal of the American Chemical Society, 1947, 69, 2907); isoxsuprine (U.S. Pat. No. 3,056,836); kallidin (Nicolaides et al., Biochem. Biophys. Res. Commun., 1961, 6, 210); kallikrein (German Patent No. 1,102,973); moxisylyte (German Patent No. 905,738); nafronyl; nicametate; nicofaranose (Swiss Patent No. 3,66,523); nylidrin (U.S. Pat. Nos. 2,661,372 and 2,661,373); pentifylline; pentoxifylline, which may be prepared as disclosed U.S. Pat. No. 3,422,107; piribedil (U.S. Pat. No. 3,299,067); prostaglandin E₁, which may be prepared by any of the methods referenced in the Merck Index, Twelfth Edition, Budaveri, Ed, New Jersey 1996, page 1353); suloctidil (German Patent No. 2,334,404); tolazoline (U.S. Pat. No. 2,161,938); and xanthinol niacinate (German Patent No. 1,102,750 or Korbonits et al, Acta. Pharm. Hung., 1968, 38, 98). The disclosures of all such patents and references are incorporated herein by reference.

The term “diuretic”, within the scope of this invention, includes but is not limited to diuretic benzothiadiazine derivatives, diuretic organomercurials, diuretic purines, diuretic steroids, diuretic sulfonamide derivatives, diuretic uracils and other diuretics such as amanozine (Austrian Patent No. 168,063); amiloride (Belguim Patent No. 639,386); arbutin (Tschitschibabin et al., Annalen, 1930, 479, 303); chlorazanil (Austrian Patent No. 168,063); ethacrynic acid (U.S. Pat. No. 3,255,241); etozolin (U.S. Pat. No. 3,072,653); hydracarbazine (British Patent No. 856,409); isosorbide (U.S. Pat. No. 3,160,641); mannitol; metochalcone (Freudenberg et al., Ber., 1957, 90, 957); muzolimine (U.S. Pat. No. 4,018,890); perhexiline; ticrynafen (U.S. Pat. No. 3,758,506); triamterene (U.S. Pat. No. 3,081,230); and urea. The disclosures of all such patents and references are incorporated herein by reference.

Diuretic benzothiadiazine derivatives within the scope of this invention include, but are not limited to: althiazide (British Patent No. 902,658); bendroflumethiazide (U.S. Pat. No. 3,392,168); benzthiazide (U.S. Pat. No. 3,440,244); benzylhydrochlorothiazide (U.S. Pat. No. 3,108,097); buthiazide (British Patents Nos. 861,367 and 885,078); chlorothiazide (U.S. Pat. Nos. 2,809,194 and 2,937,169); chlorthalidone (U.S. Pat. No. 3,055,904); cyclopenthiazide (Belguim Patent No. 587,225); cyclothiazide (Whitehead et al Journal of Organic Chemistry, 1961, 26, 2814); epithiazide (U.S. Pat. No. 3,009,911); ethiazide (British Patent No. 861,367); fenquizone (U.S. Pat. No. 3,870,720); indapamide (U.S. Pat. No. 3,565,911); hydrochlorothiazide (U.S. Pat. No. 3,164,588); hydroflumethiazide (U.S. Pat. No. 3,254,076); methyclothiazide (Close et al., Journal of the American Chemical Society, 1960, 82, 1132); meticrane (French Patents Nos. M2790 and 1,365,504); metolazone (U.S. Pat. No. 3,360,518); paraflutizide (Belguim Patent No. 15 620,829); polythiazide (U.S. Pat. No. 3,009,911); quinethazone (U.S. Pat. No. 2,976,289); teclothiazide (Close et al., Journal of the American Chemical Society, 1960, 82, 1132); and trichlormethiazide (deStevens et al., Experientia, 1960, 16, 113). The disclosures of all such patents and references are incorporated herein by reference.

Diuretic sulfonamide derivatives within the scope of this invention include, but are not limited to: acetazolamide (U.S. Pat. No. 2,554,816); ambuside (U.S. Pat. No. 3,188,329); azosemide (U.S. Pat. No. 3,665,002); bumetanide (U.S. Pat. No. 3,806,534); butazolamide (British Patent No. 769,757); chloraminophenamide (U.S. Pat. Nos. 2,909,194, 2,965,655 and 2,965,656); clofenamide (Olivier, Rec. Trav. Chim., 1918, 37, 307); clopamide (U.S. Pat. No. 3,459,756); clorexolone (U.S. Pat. No. 3,183,243); disulfamide (British Patent No. 851,287); ethozolamide (British Patent No. 795,174); furosemide (U.S. Pat. No. 3,058,882); mefruside (U.S. Pat. No. 3,356,692); methazolamide (U.S. Pat. No. 2,783,241); piretanide (U.S. Pat. No. 4,010,273); torsemide (U.S. Pat. No. 4,018,929); tripamide (Japanese Patent No. 7305,585); and xipamide (U.S. Pat. No. 3,567,777). The disclosures of all such patents and references are incorporated herein by reference.

Further, the anti-hypertensive agents which may be used in accordance with this invention and the pharmaceutically acceptable salts thereof may occur as prodrugs, hydrates or solvates. Said hydrates and solvates are also within the scope of the present invention.

Preferred anti-hypertensive agents of the invention include calcium channel blockers, alpha-adrenergic blockers, and ACE inhibitors.

The anti-hypertensives described herein are generally commercially available, or they may be made by standard techniques including those described in the references given hereinbefore.

The combination of a compound of formula 1 and the taxane derivative, the platinum coordination complex, the nucleoside analog, or the anthracycline, may be additive or syngergistic. Preferably the combination of a compound of formula 1 and the taxane derivative, the platinum coordination complex, the nucleoside analog, or the anthracycline are synergistic or exhibit a synergism. Synergism or synergistic as used to describe the compositions and methods of the present invention means a greater than additive biological effect. Thus, to state that cytotoxic (i.e., the taxane derivative, the platinum coordination complex, the nucleoside analog, or the anthracycline) is synergistic with compound of formula 1 means that the combination, in any form, produces greater cytotoxicity that either drug alone. In a preferred embodiment, the combinations of the present invention have a synergy of greater than 2 fold compared to each compound administered alone. In a more preferred embodiment, the combinations of the present invention have a synergy of greater than 4 fold compared to each compound administered alone.

The compounds of the present invention may have asymmetric carbon atoms. Such diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomer mixtures and pure enantiomers are considered as part of the invention.

The compounds of formula 1 that are basic in nature are capable of forming a wide variety of different salts with various inorganic and organic acids. Although such salts must be pharmaceutically acceptable for administration to animals, it is often desirable in practice to initially isolate the compound of formula 1 from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of formula 1 that are acidic in nature, are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of formulas 1. Such non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

Included in the present invention are compounds identical to the compounds of formula 1 but for the fact that one or more hydrogen or carbon atoms are replaced by isotopes thereof. Such compounds are useful as research and diagnostic tools in metabolism pharmokinetic studies and in binding assays. Specific applications in research include radioligand binding assays, autoradiography studies and in vivo binding studies. Included among the radiolabelled forms of the compounds of formula 1 are the tritium and C¹⁴ isotopes thereof.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), intraocular, intraperitoneal, intravesicular, intravaginal, topical, and rectal administration.

The amount of each of the active compounds administered will be dependent on the subject being treated, the severity of the disorder or condition, the rate of administration and the judgement of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The products of which the combination are composed may be administered simultaneously, separately or spaced out over a period of time so as to obtain the maximum efficacy of the combination; it being possible for each administration to vary in its duration from a rapid administration to a continuous perfusion. As a result, for the purposes of the present invention, the combinations are not exclusively limited to those which are obtained by physical association of the constituents, but also to those which permit a separate administration, which can be simultaneous or spaced out over a period of time. The compositions according to the invention are preferably compositions which can be administered parentally. However, these compositions may be administered orally or intraperitoneally in the case of localized regional therapies.

The compositions for parental administration are generally pharmaceutically acceptable, sterile solutions or suspensions which may optionally be prepared as required at the time of use. For the preparation of non-aqueous solutions or suspensions, natural vegetable oils such as—olive oil, sesame oil or liquid petroleum or injectable organic esters such as ethyl oleate may be used. The sterile aqueous solutions can consist of a solution of the product in water. The aqueous solutions are suitable for intravenous administration provided the pH is appropriately adjusted and the solution is made isotonic, for example with a sufficient amount of sodium chloride or glucose. The sterilization may be carried out by heating or by any other means which does not adversely affect the composition.

The combinations may also take the form of liposomes or the form of an association with carriers as cyclodextrins or polyethylene glycols. The compositions for oral or intraperitoneal administration are preferably aqueous suspensions or solutions.

The combinations of the present invention are formulated alone, however they may also be formulated together if desired. This facilitates the easy of use (i.e., less tablets for a patient to swallow) and patient compliance since one tablet is a desired dosage form.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration, as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the compounds of the present invention and methods of preparing such compounds. It is to be understood that the scope of the present invention is not limited in any way by the scope of the following examples and preparations. The following abbreviations are used in the Examples and have the definitions indicated unless otherwise noted: QD is once a day; BID is twice a day; Q3d×4 is once every 3 days for a total of 4 treatments; FDS is fetal bovine serum; ul is microliter; pen/strep is penicillin/streptomycin; s.c. is subcutaneous; and po is by mouth.

### EXAMPLE 1

### Anti-Tumor Efficacy of Gemcitabine Hydrochloride and mesylate salt of 3-(4-Bromo-2,6-difluoro-benzyloxy)-5-[3-(4-pyrrolidin-1-yl-butyl)-ureido}-isothiazole-4-carboxylic acid amide Against the Human Pancreatic Carcinoma Capan-1

Exponentially growing Capan-1 (RPMI 1640 with 10% FBS, and pen/strep (Gibco) were harvested and inoculated s.c. (10⁷ cells/mouse, 200 μl) into the right flank of female Nu/Nu mice (˜20 grams; Charles River Laboratories, MA). 7 days after inoculation, animals with tumor approximately 150 mm³ in size were separated into groups of 11 groups of 10 animals each. Gemcitabine hydrochloride (Gemzar®) (Eli Lilly and Company, Indianapolis, Ind.) was formulated in 0.9% saline and compound X (the mesylate salt of 3-(4-Bromo-2,6-difluoro-benzyloxy)-5-[3-(4-pyrrolidin-1-yl-butyl)-ureido}-isothiazole-4-carboxylic acid amide) was formulated in 5% Gelucire (Gattefosse Inc., France).

An overview of each of the groups and the treatment is set forth below in the table.

| | | Treatment Dosage and |
|---|---|---|
| Group # | Compound(s) | Administration Procedure |
| 1 | Vehicle | 5% Gelucire, po, qd × 13 |
| 2 | Compound X | 25 mg/kg, po, qd × 13 |
| 3 | Compound X | 100 mg/kg, po, qd × 13 |
| 4 | Gemcitabine HCl | 1 mg/kg, ip, q3d × 4 |
| 5 | Gemcitabine HCl | 5 mg/kg, ip, q3d × 4 |
| 6 | Gemcitabine HCl | 80 mg/kg, ip, q3d × 4 |
| 7 | Gemcitabine HCl | Gemcitabine HCl 1 mg/kg, ip, q3d × 4 + |
| | and Compound X | Compound X 25 mg/kg, po, qd × 13 |
| 8 | Gemcitabine HCl | Gemcitabine HCl 1 mg/kg, ip, q3d × 4 + |
| | and Compound X | Compound X, 100 mg/kg, po, qd × 13 |
| 9 | Gemcitabine HCl | Gemcitabine HCl 5 mg/kg, ip, q3d × 4 + |
| | and Compound X | Compound X, 25 mg/kg, po, qd × 13 |
| 10 | Gemcitabine HCl | Gemcitabine HCl 5 mg/kg, ip, q3d × 4 + |
| | and Compound X | Compound X, 100 mg/kg, po, qd × 13 |
| 11 | Gemcitabine HCl | Gemcitabine HCl 80 mg/kg, ip, q3d × 4 + |
| | and Compound X | Compound X, 100 mg/kg, po, qd × 13 |

Animal body weight and tumor measurements were obtained at regular intervals. Tumor volume (mm³) was calculated using the formula length (mm)×width (mm)×width (mm)×0.5. The following table shows the percentage (%) inhibition of tumor growth for each of the treatments.

| | % Tumor Growth |
|---|---|
| Group # | Inhibition |
| 1 | 0 |
| 2 | 16 |
| 3 | 46 |
| 4 | 0 |
| 5 | 26 |
| 6 | 83 |
| | 66% regression* |
| 7 | 86 |
| | 69% regression* |
| 8 | 96 |
| | 89% regression* |
| 9 | 67 |
| | 16% regression* |
| 10 | 88 |
| | 60% regression* |
| 11 | 91 |
| | 67% regression* |

*Mean tumor volume on day 1 of individual group was used as 100% for the calculation of mean tumor regression.

Mean tumor volume in Gelucire vehicle group on day 13 was used for % growth inhibition calculation.

### EXAMPLE 2

### The Anti-tumor Efficacy of Paclitaxel (Taxol®) with the mesylate salt of 3-(4-Bromo-2,6-difluoro-benzyloxy)-5-[3-(4-pyrrolidin-1-yl-butyl)-ureido}-isothiazole-4-carboxylic acid amide Against the Human Non Small Cell Lung Carcinoma EBC-1

Exponentially grown human non-small cell lung carcinoma EBC-1 cells [(RPMI 1640 with 10% FBS, and pen/strep (Gibco)] were harvested and inoculated s.c. (10⁷ cells/mouse, 200 μl) into the right flank of female Nu/Nu mice (˜20 grams; Charles River Laboratories, MA.). 7 days after inoculation, tumor-bearing animals of approximately 150 mm³ in size were separated into groups of 5 animals each.

Compound X (the mesylate salt of 3-(4-Bromo-2,6-difluoro-benzyloxy)-5-[3-(4-pyrrolidin-1-yl-butyl)-ureido}-isothiazole-4-carboxylic acid amide) was formulated in 5% Gelucire (Gattefosse Inc. France) and dosed po., qd×15 at 12.5 and 100 mg/kg. Taxol® (MeadJohnson Oncology Products, Princeton, N.J.) was formulated in 0.9% sterile saline and dosed ip., qd×5 at 20 mg/kg. For control purposes one group received 5% Gelucire only (200 μl/animal, po, qd×15).

Animal body weight and tumor measurements were obtained at regular intervals. Tumor volume (mm³) was calculated using the formula length (mm)×width (mm)×width (mm)×0.5.

The following table shows the % growth tumor inhibition in test animals and tumor growth in control animals.

| | % Tumor Growth |
|---|---|
| Compound(s) Administered | Inhibition |
| Control - | 0 |
| Vehicle 5% Gelucire, po, qd × 15 | |
| Taxol ® 20 mg/kg, ip, qd × 5 | 75 |
| Compound X 12.5 mg/kg, po, qd × 15 | 34 |
| Compound X 100 mg/kg, po, qd × 15 | 72 |
| Taxol ® 20 mg/kg, ip, qd × 5 and | 91 |
| Compound X 12.5 mg/kg, po, qd × 15 | 70% regression* |
| Taxol ® 20 mg/kg, ip, qd × 5 and | 88 |
| Compound X 100 mg/kg, po, qd × 15 | 39% regression* |

*Mean tumor volume on day 1 of the individual group was used as 100% for the calculation of tumor regression.

Mean tumor volume in Gelucire vehicle group on day 15 was used for the % growth inhibition calculation.

### EXAMPLE 3

### The Anti-tumor Efficacy of Carboplatin with the mesylate salt of 3-(4-Bromo-2,6-difluoro-benzyloxy)-5-[3-(4-pyrrolidin-1-yl-butyl)-ureido}-isothiazole-4-carboxylic acid amide Against the Human Non Small Cell Lung Carcinoma EBC-1

Exponentially grown human non-small cell lung carcinoma EBC-1 cells [RPMI 1640 with 10% FBS, and pen/strep (Gibco)] were harvested and inoculated subcutaneously (10⁷ cells/mouse, 200 μl) into the right flank of female Nu/Nu mice (˜20 grams; Charles River Laboratories, MA). 7 days after inoculation, tumor-bearing animals of approximately 175 mm³ in size were separated into groups of 8 animals each.

Carboplatin (Bristol Oncology Products, Princeton, N.J.) was formulated in 0.9% saline and dosed ip., q3d×4 at 25 and 50 mg/kg. One group received 0.9% saline only (200 μl/animal, ip, q3d×4) which served as the control for the experiment. The mesylate salt of 3-(4-Bromo-2,6-difluoro-benzyloxy)-5-[3-(4-pyrrolidin-1-yl-butyl)-ureido}-isothiazole-4-carboxylic acid amide was formulated in 5% Gelucire (Gattefosse Inc., France) and dosed po, qd×14 at 100 mg/kg. One group received 5% Gelucire only, (200 μl/animal, po, qd×14) which served as the vehicle control for the experiment.

Animal body weight and tumor measurements were obtained at regular intervals. Tumor volume (mm³) was calculated using the formula length (mm)×width (mm)×width (mm)×0.5.

Carboplatin (25 or 50 mg/kg, ip, q3d×4) co-administered with the mesylate salt of 3-(4-Bromo-2,6-difluoro-benzyloxy)-5-[3-(4-pyrrolidin-1-yl-butyl)-ureido}-isothiazole-4-carboxylic acid amide at 100 mg/kg (po, qd×14) was well tolerated and no mortality occurred.

The following table shows the treatments for each of the groups of 8 experimental animals and the second column shows the % tumor growth.

| | % Tumor Growth |
|---|---|
| Group Treatments | Inhibition |
| Control - | 41 |
| Vehicle 5% Gelucire, po, qd × 14 | |
| Control - | 0 |
| Vehicle 0.9% Saline, ip, q3d × 4 | |
| Carboplatin 25 mg/kg, ip, q3d × 4 | 0 |
| Carboplatin 50 mg/kg, ip, q3d × 4 | 31 |
| Compound X 100 mg/kg, po, qd × 4 | 67 |
| Carboplatin 25 mg/kg, ip, q3d × 4, | 78 |
| And Compound X 100 mg/kg, po, qd × 14 | 30% regression* |
| Carboplatin 50 mg/kg, ip, q3d × 4, | 76 |
| And Compound X 100 mg/kg, po, qd × 14 | 30% regression* |

*Mean tumor volume on day 1 of the individual group was used as 100% for the calculation of tumor regression.

Mean tumor volume in saline vehicle group on day 14 was used for the % growth inhibition calculation.

A similar experiment was conducted using carboplatin and mesylate salt of 3-(4-Bromo-2,6-difluoro-benzyloxy)-5-[3-(4-pyrrolidin-1-yl-butyl)-ureido}-isothiazole-4-carboxylic acid amide in against the Human Pancreatic Carcinoma Capan-1 and no negative interactions were observed.

### EXAMPLE 4

### The Anti-tumor Efficacy of 5-FU (5 flurouracil) with the mesylate salt of 3-(4-Bromo-2,6-difluoro-benzyloxy)-5-[3-(4-pyrrolidin-1-yl-butyl)-ureido}-isothiazole-4-carboxylic acid amide Against the Human Colon Carcinoma Colo-205

Exponentially grown human colon carcinoma Colo-205 cells [RPMI 1640 with 10% FBS, and pen/strep (Gibco)] were harvested and inoculated subcutaneously (5×10⁶ cells/mouse, 200 μl) into the right flank of female Nu/Nu mice (˜20 grams; Charles River Laboratories, MA). 9 days after inoculation, tumor-bearing animals of approximately 150 mm³ in size were separated into groups of 7 animals each.

5-FU (ICN Pharmaceuticals, Inc., Costa Mesa, Calif.) was formulated in 0.9% saline and dosed iv×1 at 25 and 100 mg/kg. One group received 0.9% saline only (200 μl/animal, iv×1) which served as the control for the experiment. The mesylate salt of 3-(4-Bromo-2,6-difluoro-benzyloxy)-5-[3-(4-pyrrolidin-1-yl-butyl)-ureido}-isothiazole-4-carboxylic acid amide was formulated in 5% Gelucire (Gattefosse Inc., France) and dosed po, qd×12 at 25, 50 and 100 mg/kg. One group received 5% Gelucire only, (200 μl/animal, po, qd×12) which served as the vehicle control for the experiment.

Animal body weight and tumor measurements were obtained at regular intervals. Tumor volume (mm³) was calculated using the formula length (mm)×width (mm)×width (mm)×0.5.

5-FU co-administered with the mesylate salt of 3-(4-Bromo-2,6-difluoro-benzyloxy)-5-[3-(4-pyrrolidin-1-yl-butyl)-ureido}-isothiazole-4-carboxylic acid amide at 100 mg/kg (po, qd×12) was well tolerated and no mortality occurred.

The following table shows the treatments for each of the groups of 7 experimental animals and the second column shows the % tumor growth.

| | % Tumor Growth |
|---|---|
| Group Treatments | Inhibition |
| Control - | 0 |
| Vehicle 5% Gelucire, po, qd × 12 | |
| Control - | 24 |
| Vehicle 0.9% Saline, iv × 1 | |
| 5-FU 25 mg/kg, iv × 1 | 24 |
| 5-FU 100 mg/kg, iv × 1 | 59 |
| Compound X 25 mg/kg, po, qd × 12 | 21 |
| Compound X 50 mg/kg, po, qd × 12 | 47 |
| Compound X 100 mg/kg, po, qd × 12 | 64 |
| 5-FU 25 mg/kg, iv × 1 and Compound X | 32 |
| 25 mg/kg, po, qd × 12 | |
| 5-FU 25 mg/kg, iv × 1 and Compound X | 70 |
| 50 mg/kg, po, qd × 12 | |
| 5-FU 100 mg/kg, iv × 1 and Compound X | 79 |
| 100 mg/kg, po, qd × 12 | 1% Regression* |

*Mean tumor volume on day 1 of the individual group was used as 100% for the calculation of tumor regression.

Mean tumor volume in Gelucire vehicle group on day 12 was used for the % growth inhibition calculation.

### EXAMPLE 5

### The Anti-tumor Efficacy of CPT-11 (Camptosar®) with the mesylate salt of 3-(4-Bromo-2,6-difluoro-benzyloxy)-5-[3-(4-pyrrolidin-1-yl-butyl)-ureido}-isothiazole-4-carboxylic acid amide Against the Human Colon Carcinoma Colo-205

Exponentially grown human colon carcinoma Colo-205 cells [RPMI 1640 with 10% FBS, and pen/strep (Gibco)] were harvested and inoculated subcutaneously (5×10⁶ cells/mouse, 200 μl) into the right flank of female Nu/Nu mice (˜20 grams; Charles River Laboratories, MA). 9 days after inoculation, tumor-bearing animals of approximately 150 mm³ in size were separated into groups of 7 animals each.

CPT-11 (Camptosar®, Pharmacia & Upjohn, Kalamazoo, Mich.) was formulated in 0.9% saline and dosed iv×1 at 25 and 75 mg/kg. One group received 0.9% saline only (200 μl/animal, iv×1) which served as the control for the experiment. The mesylate salt of 3-(4-Bromo-2,6-difluoro-benzyloxy)-5-[3-(4-pyrrolidin-1-yl-butyl)-ureido}-isothiazole-4-carboxylic acid amide was formulated in 5% Gelucire (Gattefosse Inc., France) and dosed po, qd×12 at 25, 50 and 100 mg/kg. One group received 5% Gelucire only, (200 μl/animal, po, qd×12) which served as the vehicle control for the experiment.

Animal body weight and tumor measurements were obtained at regular intervals. Tumor volume (mm³) was calculated using the formula length (mm)×width (mm)×width (mm)×0.5.

CPT-11 (75 mg/kg, iv×1) co-administered with the mesylate salt of 3-(4-Bromo-2,6-difluoro-benzyloxy)-5-[3-(4-pyrrolidin-1-yl-butyl)-ureido}-isothiazole-4-carboxylic acid amide at 100 mg/kg was well tolerated and no mortality occurred. The combination of 3-(4-Bromo-2,6-difluoro-benzyloxy)-5-[3-(4-pyrrolidin-1-yl-butyl)-ureido}-isothiazole-4-carboxylic acid amide and CPT-11 was well tolerated. The following table shows the results of change in tumor % growth in control and experimental animals.

The following table shows the treatments for each of the groups of 7 experimental animals and the second column shows the % tumor growth.

| | % Tumor Growth |
|---|---|
| Group Treatments | Inhibition (Growth) |
| Control - | 0 |
| Vehicle 5% Gelucire, po, qd × 12 | |
| Control - | (73% Tumor Growth) |
| Vehicle 0.9% Saline, iv × 1 | |
| CPT-11 25 mg/kg, iv × 1 | (7% Tumor Growth) |
| CPT-11 75 mg/kg, iv × 1 | 5 |
| Compound X 25 mg/kg, po, qd × 12 | 9 |
| Compound X 50 mg/kg, po, qd × 12 | 28 |
| Compound X 100 mg/kg, po, qd × 12 | 66 |
| CPT-11 25 mg/kg, iv × 1 and Compound X | 9 |
| 25 mg/kg, po, qd × 12 | |
| CPT-11 25 mg/kg, iv × 1 and Compound X | 33 |
| 50 mg/kg, po, qd × 12 | |
| CPT-11 75 mg/kg, iv × 1 once and | 58 |
| Compound X 100 mg/kg, po, qd × 12 | |

Mean tumor volume in Gelucire vehicle group on day 12 was used for the % growth inhibition calculation.

### EXAMPLE 6

### The Anti-tumor Efficacy of Doxorubicin with the mesylate salt of 3-(4-Bromo-2,6-difluoro-benzyloxy)-5-[3-(4-pyrrolidin-1-yl-butyl)-ureido}-isothiazole-4-carboxylic acid amide Against the Human Breast Carcinoma MDA-MB-231

Exponentially grown human breast carcinoma MDA-MB-231 cells [DMEM with 10% FBS, and pen/strep (Gibco)] were harvested and inoculated subcutaneously (3-5×10⁶ cells/mouse, 200 μl) into the right flank of female Nu/Nu mice (˜20 grams; Charles River Laboratories, MA). 36 days after inoculation, tumor-bearing animals of approximately 75 mm³ in size were separated into control (n=19) or experimental groups of animals (n=7 each).

Doxorubicin (Pharmacia & Upjohn, Kalamazoo, Mich.) was formulated in 0.9% saline and dosed ip×1 at 2.5 and 100 mg/kg. The mesylate salt of 3-(4-Bromo-2,6-difluoro-benzyloxy)-5-[3-(4-pyrrolidin-1-yl-butyl)-ureido}-isothiazole-4-carboxylic acid amide was formulated in 5% Gelucire (Gattefosse Inc., France) and dosed po, qd×24 at 12.5, 25, 50 and 100 mg/kg. One group received 5% Gelucire only, (200 μl/animal, po, qd×24) which served as the vehicle control for the experiment.

Animal body weight and tumor measurements were obtained at regular intervals. Tumor volume (mm³) was calculated using the formula length (mm)×width (mm)×width (mm)×0.5.

Doxorubicin (2.5 mg/kg, ip×1) co-administered with the mesylate salt of 3-(4-Bromo-2,6-difluoro-benzyloxy)-5-[3-(4-pyrrolidin-1-yl-butyl)-ureido}-isothiazole-4-carboxylic acid amide≦100 mg/kg (po, qd×24) was well tolerated and no mortality occurred. The higher dose of Doxorubicin (10 mg/kg, ip×1) co-administered with the mesylate salt of 3-(4-Bromo-2,6-difluoro-benzyloxy)-5-[3-(4-pyrrolidin-1-yl-butyl)-ureido}-isothiazole-4-carboxylic acid amide (25 or 100 mg/kg, po, qd×24) caused animal mortality.

The following table shows the treatments for each of the groups of 7 experimental animals and the second column shows the % tumor growth.

| | % Tumor Growth |
|---|---|
| Group Treatments | Inhibition |
| Control | 0 |
| Vehicle 5% Gelucire, po, qd × 24 | |
| Doxorubicin 2.5 mg/kg, ip × 1 | 7 |
| Doxorubicin 10 mg/kg, ip × 1 | 48 |
| Compound X 12.5 mg/kg, po, qd × 24 | (10% tumor growth) |
| Compound X 25 mg/kg, po, qd × 24 | 24 |
| Compound X 50 mg/kg, po, qd × 24 | 46 |
| Compound X 100 mg/kg, po, qd × 24 | 49 |
| Doxorubicin 2.5 mg/kg, ip, qd × 1 and | 36 |
| Compound X 12.5 mg/kg, po, qd × 24 | |
| Doxorubicin 2.5 mg/kg, ip, qd × 1 and | 28 |
| Compound X 25 mg/kg, po, qd × 24 | |
| Doxorubicin 2.5 mg/kg, ip, qd × 1 and | 44 |
| Compound X 100 mg/kg, po, qd × 24 | |
| Doxorubicin 10 mg/kg, ip, qd × 1 and | 45 |
| Compound X 12.5 mg/kg, po, qd × 24 | |
| Doxorubicin 10 mg/kg, ip, qd × 1 and | 67 |
| Compound X 25 mg/kg, po, qd × 24 | 5% regression* |

*Mean tumor volume on day 1 of the individual group was used as 100% for the calculation of tumor regression.

Mean tumor volume in Gelucire vehicle group on day 24 was used for the % growth inhibition calculation.

1. A method of treating a hyperproliferative disorder in a mammal which comprises administering to said mammal in need of such treatment, either simultaneously or sequentially, (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of a compound of the formula 1 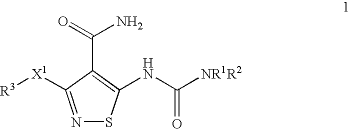
or a pharmaceutically acceptable salt, prodrug or solvate thereof, wherein:
wherein X¹ is O or S;
R¹ is H, C₁-C₁₀ alkyl, C₂-C₁₀ alkenyl, C₂-C₁₀ alkynyl, —C(O)(C₁-C₁₀ alkyl), —(CH₂)ₜ(C₆-C₁₀ aryl), —(CH₂)ₜ(4-10 membered heterocyclic), —C(O)(CH₂)ₜ(C₆-C₁₀ aryl), or —C(O)(CH₂)ₜ(5-10 membered heterocyclic), wherein t is an integer from 0 to 5; said alkyl group optionally includes 1 or 2 hetero moieties selected from O, S and —N(R⁶)— with the proviso that two O atoms, two S atoms, or an O and S atom are not attached directly to each other; said aryl and heterocyclic R¹ groups are optionally fused to a C₆-C₁₀ aryl group, a C₅-C₈ saturated cyclic group, or a 5-10 membered heterocyclic group; 1 or 2 carbon atoms in the foregoing heterocyclic moieties are optionally substituted by an oxo (═O) moiety; the —(CH₂)ₜ— moieties of the foregoing R¹ groups optionally include a carbon-carbon double or triple bond where t is an integer from 2 to 5; and the foregoing R¹ groups, except H, are optionally substituted by 1 to 3 R⁴ groups;
R² is selected from the list of substituents provided in the definition of R¹, —SO₂(CH₂)ₜ(C₆-C₁₀ aryl), —SO₂(CH₂)ₜ(5-10 membered heterocyclic), and —OR⁵, t is an integer ranging from 0 to 5, the —(CH₂)ₜ— moieties of the foregoing R² groups optionally include a carbon-carbon double or triple bond where t is an integer from 2 to 5, and the foregoing R² groups are optionally substituted by 1 to 3 R⁴ groups;
or R¹ and R² may be taken together with the nitrogen to which each is attached to form a 4-10 membered saturated monocyclic or polycyclic ring or a 5-10 membered heteroaryl ring, wherein said saturated and heteroaryl rings optionally include 1 or 2 heteroatoms selected from O, S and —N(R⁶)— in addition to the nitrogen to which R¹ and R² are attached, said —N(R⁶)— is optionally ═N— or —N═ where R¹ and R² are taken together as said heteroaryl group, said saturated ring optionally may be partially unsaturated by including 1 or 2 carbon-carbon double bonds, and said saturated and heteroaryl rings, including the R⁶ group of said —N(R⁶)—, are optionally substituted by 1 to 3 R⁴ groups;
R³ is H, C₁-C₁₀ alkyl, C₂-C₁₀ alkenyl, C₂-C₁₀ alkynyl, —(CH₂)ₜ(C₆-C₁₀ aryl), or —(CH₂)ₜ(5-10 membered heterocyclic), wherein t is an integer from 0 to 5; said alkyl group optionally includes 1 or 2 hetero moieties selected from O, S and —N(R⁶)— with the proviso that two O atoms, two S atoms, or an O and S atom are not attached directly to each other; said aryl and heterocyclic R³ groups are optionally fused to a C₆-C₁₀ aryl group, a C₅-C₈ saturated cyclic group, or a 5-10 membered heterocyclic group; 1 or 2 carbon atoms in the foregoing heterocyclic moieties are optionally substituted by an oxo (═O) moiety, the —(CH₂)ₜ— moieties of the foregoing R³ groups optionally include a carbon-carbon double or triple bond where t is an integer from 2 to 5, and the foregoing R³ groups are optionally substituted by 1 to 5 R⁴ groups;
each R⁴ is independently selected from C₁-C₁₀ alkyl, C₂-C₁₀ alkenyl, C₂-C₁₀ alkynyl, halo, cyano, nitro, trifluoromethyl, trifluoromethoxy, azido, —OR⁵, —C(O)R⁵, —C(O)OR⁵, —NR⁶C(O)OR⁵, —OC(O)R⁵, —NR⁶SO₂R⁵, —SO₂NR⁵R⁶, —NR⁶C(O)R⁵, —C(O)NR⁵R⁶, —NR⁵R⁶, —S(O)ⱼR⁷ wherein j is an integer ranging from 0 to 2, —SO₃H, —NR⁵(CR⁶R⁷)ₜOR⁶, —(CH₂)ₜ(C₆-C₁₀ aryl), —SO₂(CH₂)ₜ(C₆-C₁₀ aryl), —S(CH₂)ₜ(C₆-C₁₀ aryl), —O(CH₂)ₜ(C₆-C₁₀ aryl), —(CH₂)ₜ(5-10 membered heterocyclic), and —(CR⁶R⁷)ₘOR⁶, wherein m is an integer from 1 to 5 and t is an integer from 0 to 5; said alkyl group optionally contains 1 or 2 hetero moieties selected from O, S and —N(R⁶)— with the proviso that two O atoms, two S atoms, or an O and S atom are not attached directly to each other; said aryl and heterocyclic R⁴ groups are optionally fused to a C₆-C₁₀ aryl group, a C₅-C₈ saturated cyclic group, or a 5-10 membered heterocyclic group; 1 or 2 carbon atoms in the foregoing heterocyclic moieties are optionally substituted by an oxo (═O) moiety; and the alkyl, aryl and heterocyclic moieties of the foregoing R⁴ groups are optionally substituted by 1 to 3 substituents independently selected from halo, cyano, nitro, trifluoromethyl, trifluoromethoxy, azido, —NR⁶SO₂R⁵, —SO₂NR⁵R⁶, —C(O)R⁵, —C(O)OR⁵, —OC(O)R⁵, —NR⁶C(O)R⁵, —C(O)NR⁵R⁶, —NR⁵R⁶, —(CR⁶R⁷)ₘOR⁶ wherein m is an integer from 1 to 5, —OR⁵ and the substituents listed in the definition of R⁵;
each R⁵ is independently selected from H, C₁-C₁₀ alkyl, —(CH₂)ₜ(C₆-C₁₀ aryl), and —(CH₂)ₜ(5-10 membered heterocyclic), wherein t is an integer from 0 to 5; said alkyl group optionally includes 1 or 2 hetero moieties selected from O, S and —N(R⁶)— with the proviso that two O atoms, two S atoms, or an O and S atom are not attached directly to each other; said aryl and heterocyclic R⁵ groups are optionally fused to a C₆-C₁₀ aryl group, a C₅-C₈ saturated cyclic group, or a 5-10 membered heterocyclic group; and the foregoing R⁵ substituents, except H, are optionally substituted by 1 to 3 substituents independently selected from halo, cyano, nitro, trifluoromethyl, trifluoromethoxy, azido, —C(O)R⁶, —C(O)OR⁶, —CO(O)R⁶, —NR⁶C(O)R⁷, —C(O)NR⁶R⁷, —NR⁶R⁷, hydroxy, C₁-C₆ alkyl, and C₁-C₆ alkoxy; and
each R⁶ and R⁷ is independently H or C₁-C₆ alkyl.

2. The method of claim 1, wherein the taxane is selected from the group consisting of paclitaxel and docetaxel.

3. The method of claim 2, wherein the taxane is paclitaxel.

4. The method of claim 2, wherein the taxane is docetaxel.

5. The method according to claim 1, wherein the nucleoside analog is gemcitabine hydrochloride.

6. The method according to claim 1, wherein the platinum coordination complex is selected from the group consisting of carboplatin and tetraplatin.

7. The method according to claim 6, wherein the platinum coordination complex is carboplatin.

8. The method according to claim 7, wherein the platinum coordination complex is tetraplatin.

9. The method according to claim 1, wherein the nucleoside analog is gemcitabine hydrochloride.

10. The method according to claim 1, wherein the nucleoside analog is 5-FU.

11. The method according to claim 1, wherein the anthracycline is selected from the group consisting of doxorubicin, carminomycin and aclacinomycin.

12. The method according to claim 10, wherein the anthracycline is doxorubicin.

13. The method according to claim 1, wherein the topisomerase is Camptosar®.

14. The method according to claim 1, wherein the aromatase inhibitor is selected from the group consisting of letrazole, vorazole, Aromasin® (exemestane), and anastrazole.

15. The method according to claim 14, wherein the aromatase inhibitor is selected from the group consisting of Aromasin® (exemestane), and anastrazole.

16. The method according to claim 15, wherein the aromatase inhibitor is Aromasin® (exemestane).

17. The method according to claim 15, wherein the aromatase inhibitor is anastrazole.

18. The method of claim 1, wherein the hyperproliferative disorder is cancer.

19. The method of claim 18, wherein said cancer is selected from the group consisting of brain, squamous cell, bladder, gastric, pancreatic, breast, head, neck, oesophageal, prostate, colorectal, lung, renal, kidney, ovarian, gynecological and thyroid cancer.

20. The method of claim 19, wherein said cancer is selected from the group consisting of prostate, breast, lung, colon and ovarian cancer.

21. The method of claim 20, wherein said cancer is selected from the group consisting of prostate, breast, and lung cancer.

22. The method of claim 21, wherein said breast cancer is metastatic breast cancer.

23. The method of claim 21, wherein said lung cancer is non-small cell lung cancer.

24. The method of claim 1, wherein said hyperproliferative disorder is non-cancerous.

25. The method of claim 24, wherein non-cancerous hyperproliferative disorder is benign hyperplasia of the skin or prostate.

26. The method of claim 1, wherein said compounds (i) and (ii) are administered simultaneously.

27. The method of claim 1, wherein said compounds (i) and (ii) are administered sequentially.

28. The method of claim 1, wherein R² of the compound of formula 1 is H and R¹ is C₁-C₁₀ alkyl optionally substituted by 1 or 2 substituents independently selected from —NR⁵R⁶, —NR⁵(CR⁶R⁷)ₜOR⁶ and —(CH₂)ₜ(5-10 membered heterocyclic) wherein t is an integer from 0 to 5.

29. The method of claim 28, wherein R¹ of the compound of formula 1 is selected from propyl, butyl, pentyl and hexyl, and said R¹ groups are optionally substituted by dimethylamino, hydroxy, pyrrolidinyl, morpholino, and ethyl-(2-hydroxy-ethyl)-amino.

30. The method of claim 1, wherein R² of the compound of formula 1 is H and R¹ of the compound of formula 1 is —(CH₂)ₜ(5-10 membered heterocyclic), wherein t is an integer from 0 to 5; said heterocyclic group is optionally fused to a C₆-C₁₀ aryl group, a C₅-C₈ saturated cyclic group, or a 5-10 membered heterocyclic group; and said R¹ group, including the optionally fused portions of said R¹ group, is optionally substituted by 1 or 2 substituents independently selected from C₁-C₄ alkyl, hydroxy and hydroxymethyl.

31. The method of claim 30, wherein the heterocyclic moiety of said R¹ group is selected from morpholino, pyrrolidinyl, imidazolyl, piperazinyl, piperidinyl, and 2,5-diaza-bicyclo[2.2.1]hept-2-yl, the t variable of said R¹ group ranges from 2 to 5, and said R¹ group is optionally substituted by hydroxy, hydroxymethyl and methyl.

32. The method according to claim 1 wherein R³ of the compound of formula 1 is —(CH₂)ₜ(C₆-C₁₀ aryl) wherein t is an integer from 1 to 3 and said R³ group is optionally substituted by 1 to 4 R⁴ groups.

33. The method according to claim 32 wherein R³ is benzyl optionally substituted by 1 to 4 substituents independently selected from halo and C₁-C₄ alkyl.

34. The method according to claim 33 wherein R³ is benzyl substituted by 1 to 4 substituents independently selected from methyl, fluoro, chloro and bromo.

35. The method according to claim 1, wherein the compound of formula 1 is selected from the group consisting of
mesylate salt of 3-(4-Bromo-2,6-difluoro-benzyloxy)-5-[3-(4-pyrrolidin-1-yl-butyl)-ureido}-isothiazole-4-carboxylic acid amide;
5-{3-[3-(4-Methyl-piperazin-1-yl)-propyl]-ureido}-3-(2,3,6-trifluoro-4-methyl-benzyloxy)-isothiazole-4-carboxylic acid amide;
3-(4-Chloro-2,6-difluoro-benzyloxy)-5-(3-{4-[ethyl-(2-hydroxy-ethyl)-amino]-butyl}-ureido)-isothiazole-4-carboxylic acid amide;
3-(2-Fluoro-4-methyl-benzyloxy)-5-{3-[3-(4-methyl-piperazin-1-yl)-propyl]-ureido}-isothiazole-4-carboxylic acid amide;
3-(2,5-Difluoro-4-methyl-benzyloxy)-5-(3-4-[4-(2-hydroxy-ethyl)piperazin-1-yl]-butyl}-ureido)-isothiazole-4-carboxylic acid amide;
3-(2,5-Difluoro-4-methyl-benzyloxy)-5-[3-(6-dimethylamino-hexyl)-ureido]-isothiazole-4-carboxylic acid amide;
3-(2-Fluoro-4-methyl-benzyloxy)-5-[3-(5-isopropylamino-pentyl)ureido]-isothiazole-4-carboxylic acid amide;
hydrochloride salt of 3-(4-Bromo-2,6-difluoro-benzyloxy)-5-[3-(4-pyrrolidin-1-yl-butyl)-ureido}-isothiazole-4-carboxylic acid amide;
3-(4-Chloro-2,6-difluoro-benzyloxy)-5-[3-(4-pyrrolidin-1-yl-butyl)-ureido]-isothiazole-4-carboxylic acid amide;
3-(4-Chloro-2,6-difluoro-benzyloxy)-5-{3-[3-(4-methyl-piperazin-1-yl)propyl]-ureido}-isothiazole-4-carboxylic acid amide;
3-(4-Chloro-2,6-difluoro-benzyloxy)-5-{3-[-(1-methyl-pyrrolidin-2-yl)-ethyl]-ureido}-isothiazole-4-carboxylic acid amide;
3-(2,6-Difluoro-4-methyl-benzyloxy)-5-[3-(4-pyrrolidin-1-yl-butyl)ureido]-isothiazole-4-carboxylic acid amide;
3-(2,6-Difluoro-4-methyl-benzyloxy)-5-[3-{4-[4-(2-hydroxy-ethyl)-piperazin-1-yl]-butyl}-ureido)-isothiazole-4-carboxylic acid amide;
3-(4-Chloro-2,6-difluoro-benzyloxy)-5-[3-(3-hydroxy-5-pyrrolidin-1-yl)-pentyl)-ureido}-isothiazole-4-carboxylic acid amide;
3-(2,5-Difluoro-4-methyl-benzyloxy)-5-{3-[4-(3,4-dihydroxy-pyrrolidin-1-yl]-butyl]-ureido}-isothiazole-4-carboxylic acid amide;
3-(4-Chloro-2,6-difluoro-benzyloxy)-5-{3-[4-(3,4-dihydroxy-pyrrolidin-1-yl)-butyl]-ureido}-isothiazole-4-carboxylic acid amide;
3-(2,5-Difluoro-4-methyl-benzyloxy)-5-{3-[4-(2-hydroxymethyl-pyrrolidin-1-yl)-butyl]-ureido}-isothiazole-4-carboxylic acid amide;
3-(4-Chloro-2,6-difluoro-benzyloxy)-5-{3-[4-(2-hydroxymethyl-pyrrolidin-1-yl)-butyl]-ureido}-isothiazole-4-carboxylic acid amide;
3-(2,5-Difluoro-4-methyl-benzyloxy)-5-{3-[4-(3-hydroxy-pyrrolidin-1-yl)-butyl]-ureido}-isothiazole-4-carboxylic acid amide;
3-(4-Bromo-2,6-difluoro-benzyloxy)-5-[3-(4-pyrrolidin-1-yl-butyl]-ureido}-isothiazole-4-carboxylic acid amide;
3-(2,6-Difluoro-4-methyl-benzyloxy)-5-[3-(4-hydroxy-5-piperidin-1-yl-pentyl)-ureido]-isothiazole-4-carboxylic acid amide;
3-(2,5-Difluoro-4-methyl-benzyloxy)-5-{3-[4-(3-hydroxy-5-piperidin-1-yl-pentyl)-ureido]-isothiazole-4-carboxylic acid amide;
3-(4-Chloro-2,6-difluoro-benzyloxy)-5-{3-[4-(2-hydroxymethyl-piperidin-1-yl)-butyl]-ureido}-isothiazole-4-carboxylic acid amide;
3-(2,5-Difluoro-4-methyl-benzyloxy)-5-(3-{4-[ethyl-(2-hydroxy-ethyl)-amino]-butyl}-ureido)-isothiazole-4-carboxylic acid amide;
3-(4-Chloro-2,6-difluoro-benzyloxy)-5-[3-(5-hydroxy-6-piperidin-1-yl)-hexyl)-ureido}-isothiazole-4-carboxylic acid amide;
3-(4-Bromo-2,3,6-trifluoro-benzyloxy)-5-{3-[3-(4-methyl-piperazin-1-yl)-propyl]-ureido}-isothiazole-4-carboxylic acid amide;
3-(2,6-Difluoro-4-methyl-benzyloxy)-5-{3-[3-(4-methyl-piperazin-1-yl-propyl]-ureido}-isothiazole-4-carboxylic acid amide;
3-(2,6-Difluoro-4-methyl-benzyloxy)-5-[3-(3-hydroxy-5-pyrrolidin-1-yl-pentyl)-ureido]-isothiazole-4-carboxylic acid amide;
5-[3-(4-Pyrrolidin-1-yl-butyl)-ureido]-3-(2,3,6-trifluoro-4-methyl-benzyloxy)-isothiazole-4-carboxylic acid amide;
5-[3-(3-Hydroxy-5-pyrrolidin-1-yl-pentyl)-ureido]-3-(2,3,6-trifluoro-4-methyl-benzyloxy)-isothiazole-4-carboxylic acid amide;
3-(4-Chloro-2,6-difluoro-benzyloxy)-5-{3-[3-(5-methyl-2,5-diazabicyclo[2.2.1]hept-2-yl)-propyl]-ureido}-isothiazole-4-carboxylic acid amide;
3-(4-Chloro-2,3,6-trifluoro-benzyloxy)-5-{3-[3-(5-methyl-2,5-diaza-bicyclo[2.2.1]hept-2-yl)-propyl]-ureido}-isothiazole-4-carboxylic acid amide;
3-(4-Chloro-2,3,6-trifluoro-benzyloxy)-5-{3-[2-(1-methyl-pyrrolidin-2-yl)-ethyl]-ureido}-isothiazole-4-carboxylic acid amide;
3-(4-Chloro-2,3,6-trifluoro-benzyloxy)-5-[3-(4-pyrrolidin-1-yl-butyl)-ureido]-isothiazole-4-carboxylic acid amide;
3-(4-Chloro-2,3,6-trifluoro-benzyloxy)-5-{3-[4-(2-hydroxymethyl-pyrrolidin-1-yl)-butyl]-ureido}-isothiazole-4-carboxylic acid amide;
5-{3-[2-(1-Methyl-pyrrolidin-2-yl)-ethyl]-ureido}-3-(2,3,6-trifluoro-4-methyl-benzyloxy)-isothiazole-4-carboxylic acid amide;
5-[3-(4-Dimethylamino-butyl)-ureido]-3-(2,3,6-trifluoro-4-methyl-benzyloxy)-isothiazole-4-carboxylic acid amide;
5-[3-(3-Dimethylamino-propyl)-ureido]-3-(2,3,6-trifluoro-4-methyl-benzyloxy)-isothiazole-4-carboxylic acid amide;
5-[3-(3-Hydroxy-5-isopropropylamino-pentyl)-ureido]-3-(2,3,6-trifluoro-4-methyl-benzyloxy)-isothiazole-4-carboxylic acid amide;
5-[3-(3-Isopropylamino-propyl)-ureido]-3-(2,3,6-trifluoro-4-methyl-benzyloxy)-isothiazole-4-carboxylic acid amide;
5-{3-[4-(4-Methyl-piperazin-1-yl)-butyl]-ureido}-3-(2,3,6-trifluoro-4-methyl-benzyloxy)-isothiazole-4-carboxylic acid amide;
5-(3-{4-[4-(2-Hydroxy-ethyl)-piperazin-1-yl]-butyl}-ureido)-3-(2,3,6-trifluoro-4-methyl-benzyloxy)-isothiazole-4-carboxylic acid amide;
5-[3-(3-Pyrrolidin-1-yl-propyl)-ureido]-3-(2,3,6-trifluoro-4-methyl-benzyloxy)-isothiazole-4-carboxylic acid amide;
5-[3-(4-Hydroxy-5-piperidin-1-yl-pentyl)-ureido]-3-(2,3,6-trifluoro-4-methyl-benzyloxy)-isothiazole-4-carboxylic acid amide;
3-(4Chloro-2,6-difluoro-benzyloxy)-5-[3-(4-imidazol-1-yl-butyl)ureido]-isothiazole-4-carboxylic acid amide;
5-(3-{4-[Ethyl-(2-hydroxy-ethyl)-amino]-butyl}-ureido)-3-(2,3,6-trifluoro-4-methyl-benzyloxy)-isothiazole-4-carboxylic acid amide;
3-(4-Chloro-(2,3,6-trifluoro-benzyloxy)-5-{3-[4-(2-hydroxmethyl-piperidin-1-yl)-butyl]-ureido}-isothiazole-4-carboxylic acid amide;
3-(4-Chloro-2,3,6-trifluoro-benzyloxy)-5-[3-(3-hydroxy-5-pyrrolidin-1-yl-pentyl)-ureido]-isothiazole-4-carboxylic acid amide;
3-(4-Bromo-2,6-difluoro-benzyloxy)-5-{3-[3-(4-methyl-piperazin-1-yl)-propyl]-ureido}-isothiazole-4-carboxylic acid amide;
3-(2,6-Difluoro-4-methyl-benzyloxy)-5-{3-[2-(1-methyl-pyrrolidin-2-yl)-ethyl]-ureido}-isothiazole-4-carboxylic acid amide;
3-(2,6-Difluoro-4-methyl-benzyloxy)-5-[3-(4-dimethylamino-butyl)-ureido}-isothiazole-4-carboxylic acid amide;
3-(2,6-Difluoro-4-methyl-benzyloxy)-5-[3-(3-dimethylamino-propyl)-ureido]-isothiazole-4-carboxylic acid amide;
3-(4-Bromo-2,3,6-trifluoro-benzyloxy)-5-[3-(4-pyrrolidin-1-yl-butyl)-ureido]-isothiazole-4-carboxylic acid amide;
3-(4-Chloro-2,3,6-trifluoro-benzyloxy)-5-[3-(4-imidazol-1-yl-butyl)-ureido]-isothiazole-4-carboxylic acid amide;
3-(4-Chloro-2,3,6-difluoro-benzyloxy)-5-(3-{3-[ethyl-(2-hydroxy-ethyl)-amino]-propyl}-ureido)-isothiazole-4-carboxylic acid amide;
3-(4-Chloro-2,3,6-trifluoro-benzyloxy)-5-(3-{3-[ethyl-(2-hydroxy-ethyl)-amino]-propyl}-ureido)-isothiazole-4-carboxylic acid amide;
5-[3-(3-Methylamino-propyl)-ureido]-3-(2,3,6-trifluoro-4-methyl-benzyloxy)-isothiazole-4-carboxylic acid amide;
5-[3-(3-Amino-propyl)-3-methyl-ureido]-3-(2,3,6-trifluoro-4-methyl-benzyloxy)-isothiazole-4-carboxylic acid amide;
5-[3-(4-Diethylamino-butyl)-ureido]-3-(2,3,6-trifluoro-4-methyl-benzyloxy)-isothiazole-4-carboxylic acid amide;
3-(2,6-Difluoro-4-methyl-benzyloxy)-5-[3-(3-pyrrolidin-1-yl-propyl)-ureido]-isothiazole-4-carboxylic acid amide;
3-(3-Chloro-2,6-difluoro-4-methyl-benzyloxy)-5-[3-(4-dimethylamino-butyl)-ureido]-isothiazole-4-carboxylic acid amide;
5-(3-{4-[Bis-(2-hydroxy-ethyl)-amino]-butyl}-ureido)-3-(2,6-difluoro-4-methyl-benzyloxy)-isothiazole-4-carboxylic acid amide;
and the pharmaceutically acceptable salts, prodrugs and solvates of said compounds.

36. The method according to claim 35, wherein the compound of formula 1 is selected from the group consisting of:
3-(4-Bromo-2,6-difluoro-benzyloxy)-5-[3-(4-pyrrolidin-1-yl-butyl)-ureido]-isothiazole-4-carboxylic acid amide
mesylate salt of 3-(4-Bromo-2,6-difluoro-benzyloxy)-5-[3-(4-pyrrolidin-1-yl-butyl)-ureido}-isothiazole-4-carboxylic acid amide;
5-{3-[3-(4-Methyl-piperazin-1-yl)-propyl]-ureido}-3-(2,3,6-trifluoro-4-methyl-benzyloxy)-isothiazole-4-carboxylic acid amide;
3-(4-Chloro-2,6-difluoro-benzyloxy)-5-(3-{4-[ethyl-(2-hydroxy-ethyl)-amino]-butyl}-ureido)-isothiazole-4-carboxylic acid amide;
3-(2-Fluoro-4-methyl-benzyloxy)-5-{3-[3-(4-methyl-piperazin-1-yl)-propyl]-ureido}-isothiazole-4-carboxylic acid amide;
3-(2,5-Difluoro-4-methyl-benzyloxy)-5-(3-4-[4-(2-hydroxy-ethyl)-piperazin-1-yl]-butyl}-ureido)-isothiazole-4-carboxylic acid amide;
3-(2,5-Difluoro-4-methyl-benzyloxy)-5-[3-(6-dimethylamino-hexyl)-ureido]-isothiazole-4-carboxylic acid amide;
3-(2-Fluoro-4-methyl-benzyloxy)-5-[3-(5-isopropylamino-pentyl)-ureido]-isothiazole-4-carboxylic acid amide;
hydrochloride salt of 3-(4-Bromo-2,6-difluoro-benzyloxy)-5-[3-(4-pyrrolidin-1-yl-butyl)-ureido}-isothiazole-4-carboxylic acid amide;
and the pharmaceutically acceptable salts, prodrugs and solvates of said compounds.

37. The method according to claim 36, wherein the compound of formula 1 is selected from the group consisting of
3-(4-Bromo-2,6-difluoro-benzyloxy)-5-[3-(4-pyrrolidin-1-yl-butyl)-ureido]-isothiazole-4-carboxylic acid amide
mesylate salt of 3-(4-Bromo-2,6-difluoro-benzyloxy)-5-[3-(4-pyrrolidin-1-yl-butyl)-ureido}-isothiazole-4-carboxylic acid amide;
hydrochloride salt of 3-(4-Bromo-2,6-difluoro-benzyloxy)-5-[3-(4-pyrrolidin-1-yl-butyl)-ureido}-isothiazole-4-carboxylic acid amide;
and the pharmaceutically acceptable salts, prodrugs and solvates of said compounds.

38. The method according to claim 37, wherein the compound of formula 1 is hydrochloride salt of 3-(4-Bromo-2,6-difluoro-benzyloxy)-5-[3-(4-pyrrolidin-1-yl-butyl)-ureido}-isothiazole-4-carboxylic acid amide; and the pharmaceutically acceptable salts, prodrugs and solvates of said compound.

39. The method of claim 38, comprising a therapeutically effective amount of paclitaxel.

40. The method of claim 38, comprising a therapeutically effective amount of gemcitabine hydrochloride.

41. The method of claim 38, comprising a therapeutically effective amount of carboplatin.

42. The method of claim 38, comprising a therapeutically effective amount of gemcitabine hydrochloride.

43. The method of claim 38, comprising a therapeutically effective amount of doxorubicin.

44. The method according to claim 38, comprising a therapeutically effective amount of Camptosar®.

45. The method according to claim 38, comprising a therapeutically effective amount of Aromasin® (exemestane).

46. The method according to claim 38, comprising a therapeutically effective amount of anastrazole.

47. A pharmaceutical composition for the treatment of a hyperproliferative disorder in a mammal which comprises (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of a compound of the formula 1, in combination with one or more pharmaceutically acceptable carriers or vehicles.

48. The pharmaceutical composition of claim 47, wherein the taxane is selected from the group consisting of paclitaxel and docetaxel.

49. The pharmaceutical composition of claim 48, wherein the taxane is paclitaxel.

50. The pharmaceutical composition of claim 48, wherein the taxane is docetaxel.

51. The pharmaceutical composition of claim 47, wherein the nucleoside analog is gemcitabine hydrochloride.

52. The pharmaceutical composition of claim 47, wherein the platinum coordination complex is selected from the group consisting of carboplatin and tetraplatin.

53. The pharmaceutical composition of claim 52, wherein the platinum coordination complex is carboplatin.

54. The pharmaceutical composition of claim 53, wherein the platinum coordination complex is tetraplatin.

55. The pharmaceutical composition of claim 47, wherein the nucleoside analog is gemcitabine hydrochloride.

56. The pharmaceutical composition of claim 47, wherein the anthracycline is selected from the group consisting of doxorubicin, carminomycin and aclacinomycin.

57. The pharmaceutical composition of claim 47, wherein the anthracycline is doxorubicin.

58. The pharmaceutical composition of claim 47, wherein the topisomerase inhibitor is selected from the group consisting of teniposide, amsacrine, topotecan, and Camptosar®.

59. The pharmaceutical composition of claim 58, wherein the topisomerase is Camptosar®.

60. The pharmaceutical composition of claim 47, wherein the aromatase inhibitor is selected from the group consisting of letrazole, vorazole, Aromasin® (exemestane), and anastrazole.

61. The pharmaceutical composition of claim 60, wherein the aromatase inhibitor is selected from the group consisting of Aromasin® (exemestane), and anastrazole.

62. The pharmaceutical composition of claim 61, wherein the aromatase inhibitor is Aromasin® (exemestane).

63. The pharmaceutical composition of claim 62, wherein the aromatase inhibitor is anastrazole.

64. A kit comprising in a first compartment a compound of formula 1 and in a second compartment a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar® or an aromatase inhibitor.

65. The kit of claim 64, wherein the compound in the second compartment is paclitaxel.

66. The kit of claim 64, wherein the compound in the second compartment is gemcitabine hydrochloride.

67. A method of treating a hyperproliferative disorder in a mammal which comprises administering to said mammal in need of such treatment, either simultaneously or sequentially, (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of the hydrochloride salt of 3-(4-Bromo-2,6-difluoro-benzyloxy)-5-[3-(4-pyrrolidin-1-yl-butyl)-ureido}-isothiazole-4-carboxylic acid amide.

68. A method of treating a hyperproliferative disorder in a mammal which comprises administering to said mammal in need of such treatment, either simultaneously or sequentially, (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor, and (ii) a therapeutically effective amount of a compound of the formula 1 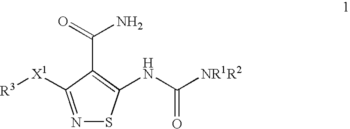
or a pharmaceutically acceptable salt, prodrug or solvate thereof, wherein:
wherein X¹ is O or S;
R¹ is H, C₁-C₁₀ alkyl, C₂-C₁₀ alkenyl, C₂-C₁₀ alkynyl, —C(O)(C₁-C₁₀ alkyl), —(CH₂)ₜ(C₆-C₁₀ aryl), —(CH₂)ₜ(4-10 membered heterocyclic), —C(O)(CH₂)ₜ(C₆-C₁₀ aryl), or —C(O)(CH₂)ₜ(5-10 membered heterocyclic), wherein t is an integer from 0 to 5; said alkyl group optionally includes 1 or 2 hetero moieties selected from O, S and —N(R⁶)— with the proviso that two O atoms, two S atoms, or an O and S atom are not attached directly to each other; said aryl and heterocyclic R¹ groups are optionally fused to a C₆-C₁₀ aryl group, a C₅-C₈ saturated cyclic group, or a 5-10 membered heterocyclic group; 1 or 2 carbon atoms in the foregoing heterocyclic moieties are optionally substituted by an oxo (═O) moiety; the —(CH₂)ₜ— moieties of the foregoing R¹ groups optionally include a carbon-carbon double or triple bond where t is an integer from 2 to 5; and the foregoing R¹ groups, except H, are optionally substituted by 1 to 3 R⁴ groups;
R² is selected from the list of substituents provided in the definition of R¹, —SO₂(CH₂)ₜ(C₆-C₁₀ aryl), —SO₂(CH₂)ₜ(5-10 membered heterocyclic), and —OR⁵, t is an integer ranging from 0 to 5, the —(CH₂)ₜ— moieties of the foregoing R² groups optionally include a carbon-carbon double or triple bond where t is an integer from 2 to 5, and the foregoing R² groups are optionally substituted by 1 to 3 R⁴ groups;
or R¹ and R² may be taken together with the nitrogen to which each is attached to form a 4-10 membered saturated monocyclic or polycyclic ring or a 5-10 membered heteroaryl ring, wherein said saturated and heteroaryl rings optionally include 1 or 2 heteroatoms selected from O, S and —N(R⁶)— in addition to the nitrogen to which R¹ and R² are attached, said —N(R⁶)— is optionally ═N— or —N═ where R¹ and R² are taken together as said heteroaryl group, said saturated ring optionally may be partially unsaturated by including 1 or 2 carbon-carbon double bonds, and said saturated and heteroaryl rings, including the R⁶ group of said —N(R⁶)—, are optionally substituted by 1 to 3 R⁴ groups;
R³ is H, C₁-C₁₀ alkyl, C₂-C₁₀ alkenyl, C₂-C₁₀ alkynyl, —(CH₂)ₜ(C₆-C₁₀ aryl), or —(CH₂)ₜ(5-10 membered heterocyclic), wherein t is an integer from 0 to 5; said alkyl group optionally includes 1 or 2 hetero moieties selected from O, S and —N(R⁶)— with the proviso that two O atoms, two S atoms, or an O and S atom are not attached directly to each other; said aryl and heterocyclic R³ groups are optionally fused to a C₆-C₁₀ aryl group, a C₅-C₈ saturated cyclic group, or a 5-10 membered heterocyclic group; 1 or 2 carbon atoms in the foregoing heterocyclic moieties are optionally substituted by an oxo (═O) moiety; the —(CH₂)ₜ— moieties of the foregoing R³ groups optionally include a carbon-carbon double or triple bond where t is an integer from 2 to 5, and the foregoing R³ groups are optionally substituted by 1 to 5 R⁴ groups;
each R⁴ is independently selected from C₁-C₁₀ alkyl, C₂-C₁₀ alkenyl, C₂-C₁₀ alkynyl, halo, cyano, nitro, trifluoromethyl, trifluoromethoxy, azido, —OR⁵, —C(O)R⁵, —C(O)OR⁵, —NR⁶C(O)OR⁵, —OC(O)R⁵, —NR⁶SO₂R⁵, —SO₂NR⁵R⁶, —NR⁶C(O)R⁵, —C(O)NR⁵R⁶, —NR⁵R⁶, —S(O)ⱼR⁷ wherein j is an integer ranging from 0 to 2, —SO₃H, —NR⁵(CR⁶R⁷)ₜOR⁶, —(CH₂)ₜ(C₆-C₁₀ aryl), —SO₂(CH₂)ₜ(C₆-C₁₀ aryl), —S(CH₂)ₜ(C₆-C₁₀ aryl), —O(CH₂)ₜ(C₆-C₁₀ aryl), —(CH₂)ₜ(5-10 membered heterocyclic), and —(CR⁶R⁷)ₘOR⁶, wherein m is an integer from 1 to 5 and t is an integer from 0 to 5; said alkyl group optionally contains 1 or 2 hetero moieties selected from O, S and —N(R⁶)— with the proviso that two O atoms, two S atoms, or an O and S atom are not attached directly to each other; said aryl and heterocyclic R⁴ groups are optionally fused to a C₆-C₁₀ aryl group, a C₅-C₈ saturated cyclic group, or a 5-10 membered heterocyclic group; 1 or 2 carbon atoms in the foregoing heterocyclic moieties are optionally substituted by an oxo (═O) moiety; and the alkyl, aryl and heterocyclic moieties of the foregoing R⁴ groups are optionally substituted by 1 to 3 substituents independently selected from halo, cyano, nitro, trifluoromethyl, trifluoromethoxy, azido, —NR⁶SO₂R⁵, —SO₂NR⁵R⁶, —C(O)R⁵, —C(O)OR⁵, —OC(O)R¹, —NR⁶C(O)R⁵, —C(O)NR⁵R⁶, —NR⁵R⁶, —(CR⁶R⁷)ₘOR⁶ wherein m is an integer from 1 to 5, —OR⁵ and the substituents listed in the definition of R⁵;
each R⁵ is independently selected from H, C₁-C₁₀ alkyl, —(CH₂)ₜ(C₆-C₁₀ aryl), and —(CH₂)ₜ(5-10 membered heterocyclic), wherein t is an integer from 0 to 5; said alkyl group optionally includes 1 or 2 hetero moieties selected from O, S and —N(R⁶)— with the proviso that two O atoms, two S atoms, or an O and S atom are not attached directly to each other; said aryl and heterocyclic R⁵ groups are optionally fused to a C₆-C₁₀ aryl group, a C₅-C₈ saturated cyclic group, or a 5-10 membered heterocyclic group; and the foregoing R⁵ substituents, except H, are optionally substituted by 1 to 3 substituents independently selected from halo, cyano, nitro, trifluoromethyl, trifluoromethoxy, azido, —C(O)R⁶, —C(O)OR⁶, —CO(O)R⁶, —NR⁶C(O)R⁷, —C(O)NR⁶R⁷, —NR⁶R⁷, hydroxy, C₁-C₆ alkyl, and C₁-C₆ alkoxy; and
each R⁶ and R⁷ is independently H or C₁-C₆ alkyl; and (iii) a therapeutically effective amount of an anti-hypertensive agent.

69. The method of claim 68, wherein the taxane is selected from the group consisting of paclitaxel and docetaxel.

70. The method of claim 69, wherein the taxane is paclitaxel.

71. The method of claim 69, wherein the taxane is docetaxel.

72. The method according to claim 68, wherein the nucleoside analog is gemcitabine hydrochloride.

73. The method according to claim 68, wherein the platinum coordination complex is selected from the group consisting of carboplatin and tetraplatin.

74. The method according to claim 73, wherein the platinum coordination complex is carboplatin.

75. The method according to claim 74, wherein the platinum coordination complex is tetraplatin.

76. The method according to claim 68, wherein the nucleoside analog is gemcitabine hydrochloride.

77. The method according to claim 68, wherein the anthracycline is selected from the group consisting of doxorubicin, carminomycin and aclacinomycin.

78. The method according to claim 68, wherein the topisomerase inhibitor is selected from the group consisting of teniposide, amsacrine, topotecan, and Camptosar®.

79. The method according to claim 78, wherein the topisomerase is Camptosar®.

80. The method according to claim 68, wherein the aromatase inhibitor is selected from the group consisting of letrazole, vorazole, Aromasin® (exemestane), and anastrazole.

81. The method according to claim 80, wherein the aromatase inhibitor is selected from the group consisting of Aromasin® (exemestane), and anastrazole.

82. The method according to claim 81 wherein the aromatase inhibitor is Aromasin® (exemestane).

83. The method according to claim 82, wherein the aromatase inhibitor is anastrazole.

84. The method according to claim 68, wherein the anti-hypertensive agent is selected from the group consisting of calcium channel blockers, angiotensin converting enzyme inhibitors (ACE inhibitors), angiotensin II receptor antagonists (A-II antagonists), diuretics, beta-adrenergic receptor blockers (β-blockers), vasodilators and alpha-adrenergic receptor blockers (α-blockers).

85. The method according to claim 84, wherein the anti-hypertensive agent is an angiotensin converting enzyme inhibitors (ACE inhibitors).

86. The method according to claim 85, wherein the ACE inhibitor is accupril (quinapril) or accuretic (quinapril with hydrochlorothiazide).

87. The method according to claim 84, wherein the anti-hypertensive agent is an alpha-adrenergic receptor blocker (α-blocker).

88. The method according to claim 87, wherein the alpha-adrenergic receptor blocker (α-blocker) is selected from the group consisting of cardura (doxazosin) or cardura XL (doxazosin GITS).

89. The method according to claim 84, wherein the anti-hypertensive agent is a calcium channel blocker.

90. The method according to claim 89, wherein the calcium channel blocker is selected from the group consisting of Norvasc (amlodipine), procardia (nifedipine) and procardia XL (nifedipine GITS).

91. A method of treating a hyperproliferative disorder in a mammal which comprises administering to said mammal in need of such treatment, either simultaneously or sequentially: (i) a therapeutically effective amount of a taxane derivative, a platinium coordination complex selected from the group consisting of carboplatin, tetraplatin, and topotecan, a nucleoside analog selected from the group consisting of gemcitabine hydrochloride and 5-FU, an anthracycline, a topoisomerase selected from the group consisting of etoposide, teniposide, amsacrine, topotecan, and Camptosar®, an aromatase inhibitor; and (ii) a therapeutically effective amount of the hydrochloride salt of 3-(4-Bromo-2,6-difluoro-benzyloxy)-5-[3-(4-pyrrolidin-1-yl-butyl)-ureido}-isothiazole-4-carboxylic acid amide; and (iii) a therapeutically effective amount an anti-hypertensive agent.

92. The method according to claim 91, wherein the anti-hypertensive agent is selected from the group consisting of calcium channel blockers, angiotensin converting enzyme inhibitors (ACE inhibitors), angiotensin II receptor antagonists (A-II antagonists), diuretics, beta-adrenergic receptor blockers (β-blockers), vasodilators and alpha-adrenergic receptor blockers (α-blockers).
